SEQUENCE LISTING (32 sequences)

SEQ ID NO: 1 - protein, 558 aa, Melanocarpus albomyces
APPEPPTNVVEKRAAPTVEISTGTIVGTTRLATEAFNGIPFALPPVGQLRLKPPVRLNSSLGVFDASGIAPACPQFLADSDSNEFLAQVINTVTGLPFFQKALKISEDCLTINVIRPKGTKAGDKLPVLFWIYGGGFELGWSSMYDGGPLVTNAIGFGKPFIFVAVNYRVAGFGFMPGKEILADGAANLGHLDQRMGLEWVADNIAAFGGDPDKVTIWGESAGAISVLNQMALFDGDHTYKGKPLFRGAIMNSGSIVPADPVDCPKGQEIYDQVVAKAGCAGASDTLACLRELPYEKFLDAANSVPAILSYNSVALSYLPRPDGKVLTKSPDVLIKEGKYAAVPMIIGDQEDEGTLFSLFQPNITNSEKLVTYLKDLFFHGASREQLEGLVDRYPARISAGSPFRTGLLNEIYPGFKRLAAILGDLVFTLTRRVFLEDATRVNPTVPAWSYLASYDYGTPILGTFHGSDLLQVFFGILPNYASRSIQSYYANFVYNLDPNDASGGTSGKSKVAEEWPRWTADDRTLIQFFANRNGYLKDDFRSEAAEYIGAHVDYLHI

SEQ ID NO: 2 - protein, 558 aa, Chaetomium thermophilum
APPEPPTKVLAKRAAPTVTISTGTVIGTDRIVTEAFNGIPFAKAPTGNLRLRPPVRINESLGVIDATGIAPACPQFLADASSNEFLAQVLDKVTNLPFFQKALKITEDCLTVSVIRPKGVKAGDKLPVLFWIYGGGFELGWSSMYDGGPLVASATSLGKPFIFVAVNYRVAGFGFMPGKEILADGAANLGLLDQRMGLEWVADNIEAFGGDPNKVTIWGESAGSISVYNQMALYDGDNTYKGKPLFRAAIMNSGSMIPADPVDCPKGQQVYNTVVAKAGCSGAADTLACLRSLPYEKFLRAANSVPAILSYNSVALSYLPRPDGKVLTKSPDLLAKEGKFAAVPMIIGDQEDEGTLFSLFQTNVTTTDRLVTYLKDIFFHNASKKQLEDLVNTYEPWVWAGSPFRTGLLNEVYPGFKRLAAILGDLVFTLTRRIFLEYVTAAKPNVPVWSYLASYDYGTPILGTFHGSDLLQVFYGVLPNYASKSIHSYYANFIYNLNPNDASGGTSSKSKVSETWPQWTSSTRKLINFFANRNEFIKDDFRSDSAAFIRNNVNILHI

SEQ ID NO: 3 - protein, 558 aa, Artificial Sequence
  synthetic sequence
APPEPPTKVLEKRAAPTVTISTGTVIGTDRIVTEAFNGIPFAKAPTGNLRLKPPVRLNESLGVIDATGIAPACPQFLADASSNEFLAQVLDKVTNLPFFQKALKISEDCLTVSVIRPKGVKAGDKLPVLFWIYGGGFELGWSSMYDGGPLVASATSLGKPFIFVAVNYRVAGFGFMPGKEILADGAANLGLLDQRMGLEWVADNIEAFGGDPNKVTIWGESAGSISVYNQMALYDGDNTYKGKPLFRGAIMNSGSMIPADPVDCPKGQQVYDTVVAKAGCSGAADTLACLRSLPYEKFLRAANSVPAILSYNSVALSYLPRPDGKVLTKSPDLLAKEGKYAAVPMIIGDQEDEGTLFSLFQTNVTTTDRLVTYLKDIFFHNASKKQLEDLVNTYEPWVWAGSPFRTGLLNEVYPGFKRLAAILGDLVFTLTRRIFLEYATAANPNVPAWSYLASYDYGTPILGTFHGSDLLQVFYGVLPNYASKSIHSYYANFVYNLDPNDASGGTSSKSKVSETWPQWTSSTRKLINFFANRNEFIKDDFRSDSAAFIRNNVNILHI

SEQ ID NO: 4 - protein, 558 aa, Myceliophthora thermophila
APPEPPTKIVEKRAAPTVTIASGSIVGVSRQATEAFNGIPFAEPPVGPLRLKPPVRLNSSLGVFDATGIAPACPQFFADSDSNDFLSRVIDTVTSLPFFQKALKVSEDCLTINVIRPKGTKAGDNLPVLFWIFGGGFELGWSSMYDGGPLVSNAIDMGKPYVFVAVNYRVGGFGFMPGKEILADGSANLGHLDQRMGLEWVADNIAAFGGDPNKVTIWGESAGAISVFNQMALYNGDNTYKGKPLFRGAIMNSGSIVPADPVDCPKGQAVYDQVVAKAGCAGASDTLDCLRQAPYDKFLAAANSVPAILSYNSVALSYLPRPDGKVITKSPDVLVKEGKYAAVPMIIGDQEDEGTLFALFQSNVTSTDKLVEYLGGLFFHGATTEQIEGLVNTYSTSILAGSPFRSGILNEIYPGFKRLAAILGDLVFTLTRRVFLEAATTVNPNVPAWSYLASYDEGTPILGTFHGSDLLQVFYGILPNYASKSIQSYYANFVYNLDPNDASGGTSAKSKVAESWPRWTGTERKLINFFSNRSGYLNDDFRSESANYISSNVGALYI

SEQ ID NO: 5 - protein, 558 aa, Corynascus thermophilus
APPEPPTNVVEKRAAPTVTIASGSIVGVNRLSTEAFNGIPFAEPPVGPLRLKPPVRLNSSLGVFDASGIAPACPQFFADSDSNEFLPKVIDFVTDLPFFQRALKISEDCLTVNVIRPKGTKAGDNLPVLFWIYGGGFELGWSSMYDGGPLVMNAINVGKPFVFVAVNYRVGGFGFMPGKEILADGSANLGHLDQRMALEWVADNIAAFGGDPNKVTIWGESAGAISVFNQMALYDGDNTYKGKPLFRGAIMNSGSIIPADPVDCPKGQAVYDQVVAKAGCAGASDTLACLRELPYDKFLSAANSVPAILSYNSVALSYLPRPDGKLITKSPDVLVKERKYAAVPMIIGDQEDEGTLFALFQSNVTNTDKLVNYLGGLFFHGATTEQIEGLVNTYPTSILAGSPFRSGIFNEIYPGFKRLAAILGDLVFTLTRRVFLETATAVNPDVPAWSYLASYDEGTPILGTFHGSDLLQVFYGILPNYASRSIQSYYANFVYNLDPNDASGGTSAKSKVAETWPRWTPNERKLINFFSNRSGFLNDDFRSESANYIGANVGVFHV

SEQ ID NO: 6 - protein, 558 aa, Myriococcum thermophilum
APPEPPSRTVEKRAAPTVTIASGSIVGVSRLATEAFNGIPFAEPPVGPLRLKPPVRLNSSLGVFDATGIAPACPQFFADTDSNEFLARVIDFVTDLPFFQKALKVSEDCLTVNVIRPKGTKAGDNLPVLFWIYGGGFELGWSSMYDGGPLVTNAIDMGKPFIFVAVNYRVGGFGFMPGKEILADGSANLGHLDQRMGLEWVADNIAAFGGDPNKVTIWGESAGAISVFNQMALYDGDNTYNGKPLFRGAIMNSGSIVPAAPVDCPKGQAVYDQVVAKAGCAGASDTLACLRELPYDKFLEAANSVPAILSYHSVALSYLPRPDGKVITQSPDILVKERKYAAVPMIIGDQEDEGTLFALFQPNITNTNKLVDYLGSLFFHEATTNQIEGLVNTYSTSILDGSPFRSGILNEIYPGFKRLAAILGDLVFTLTRRVFLETATAVNPDVPAWSYLASYDEGTPILGTFHGSDILQVFYGILPNYASRSIQSYYANFVYNLDPNDASGGTSAKSKVREDWPRWTATERKLINFFSNRSGYLKDDFRSKSAGYISENVGALYF

SEQ ID NO: 7 - protein, 559 aa, Thielavia australiensis
APPEPPSKVEEKRAAPTVSISSGTFVGVSGTTTEAFNGIPYALPPVGPLRLKPPVRLNLSLGTFDASGIAPACPQFFADSDSNDLLAQVIDTVTDLPFFQKALKISEDCLSVNVIRPKGVKAGDKLPVLFWIFGGGFELGWTSMYDAGPLVTSAVQMGKPYVFVAVNYRVGGFGFMPGKEILADGAANLGHLDQRMGLEWVADNIAAFGGDPDKVTIWGESAGAISVLNQMALYDGDNTYNGKPLFRGAIMDSGSVVPADPVDCPKGQAVYDAVVAKAGCSAAKDTLACLRALPYDEFLDAANSVPAILSYNSLALSYMPRPDGKVLTQSSDYLIKEGRYAAVPMIIGDQEDEGTLFALFQPNVTTTDDLVGYLASLFFHGATRQQIEGLVDTYSPWVWDGSPFGTGLLNEVYPGFKRTAALLGDIVFTLTRRVFLEQAVQVNPTVPAWSYLASYDFGTPVLGTFHGSDLLQVFYGLLPNCASKSIQSYYANFVYNLDPNDASGGTSGRGSKVAENWPRWTGEQRTLIRFYDDRNEYLEDDFRAEAAKYLADNADFLHV

SEQ ID NO: 8 - protein, 558 aa, Thielavia terestris
APPAPPSRVIEKRSAPTVVISSGTIVGVSREDTEAFTGIPFAQAPVGPLRLKPPVRLNSSLGTFDASQTAPACPQFLANTASDDLLSEVLTTVSNLPFFQKALKISEDCLNIDVYRPVGTKAGDNLPVLFWIFGGGFELGWSSMYDGGPLVSGAVSLGKPYILVAVNYRVGGFGFMPGKEILADGAANLGLLDQRMGLEWVADNIAAFGGDPDRVTIWGESAGAISVFDQMALYDGDNTYKGKPLFRGAIMNSGSIVPADPVDCPKGQVVYDTVVKQAGCASSQDTLACLRALPYDKFLAAANSVPAILSYSSVALSYLPRPDGKALTESPDVLVKEGKYAAVPMIIGDQEDEGTLFALFQSNITTSDKLANYLGTLFFNDASQAQVQALVGQYSPFVWDGSPFGTGLLNEVYPGFKRLAAMLGDLVFTLTRRVFLQVATSVNPSVPAWSYLASYDAGTPILGTFHGSDILQVFFGILPNYASRSIQSYYANFVYNLDPNDASGGTSAQSKVAEDWPQWTGADRNMIQFFADHSGSLVDDFRSGAADFIAANVDILHI

SEQ ID NO: 9 - protein, 558 aa, Scytalidium thermophilum
APPEPPTRVIEKRANPTVTAPAGKIIGVERVGTEAFNGIPFALPPTGSLRLKPPVRLNTSLSNFDATGIAPACPQFFADSDDKGLLSMVLDNITGLPFFQKALKVSEDCLTVNVIRPKGTKAGDKLPVLFWIYGGGFELGWSSMYDGGPIVTSAASLGKPFVFVAVNYRVGGFGFMPGKEILADGSANLGLLDQRMGLEWVADNIEAFGGDPTKVTIWGESAGAISVWNQMALYDGNNKYKGKPLFRGAIMNSGSLVPTNPVDSPKAQGIYDAVVKKAGCSSAKDTLNCLRQLSYSKFLDAANSVPHVLSYNSVALSYLPRPDGKTLTKSPELLAKEGKYAAVPMIIGDQEDEGTLFALFQPNLTSTSRLVDYFKNIYFHDAPKSKIEGLVDQYSSWVWDGSPFGSGLLYDIYPGFKRLAAILGDIVFTLTRRTFLEYATAANPNVPAWSYIASYYRGTPILGTFHASDILQVFYGVLPNYASRSIHSFYANFVYNLDPNNASGGTSSKSKVSESWPRWTAKDRKLINFFWDKSEFIKDDFREGAGKYISSNIQYLRV

SEQ ID NO: 10 - protein, 544 aa, Malbranchea cinnamomea
APEKRAASPVVTIAHPEATIIGSSALSVETFNDIPFAAPPTGPLRLKPPKPLDGALGTVDATTLIPKSCPQFYFSIDQGAIPGDILGDLMNHPLLQKVTNAGEDCLYLNVQRPRGTKAGDKLPVLFWIYGGGFQLGSTQLYNGASLVQESMRQGKPIIFVAVNYRVGGFGFLPGAEVLADGSANLGLLDQRLGLQWVADNIEAFGGDPEKVTIWGESAGAISVASHMTMYDGDHTYKGKPLFRGAIMNSGSGIPTDPVDCPKAQAVYDSVVKYAGCDTASDTLECLRGLDYEEFLDAANAVPGILSYSSVALSYLPRPDGTAFTESPDLLIEKGKYAKVPFIIGDQEDEGTLFALFQPNITTRGQIVDYLSERFFHHADKATIKGLVDTYQTISIDGSPFRTGLLNNWYPQFKRLAAILGDLTFTITRRVVLDIINRVSPEVPSWSYLATYDYGTPIMGTFHGSDILQVFHGIWPNYAARTIRGYYFNFVYNLDPNDGKLPHWPRWSEKRQLAEFQAHRVRLLADNFRSDTYDYLVKNIKKFYY

SEQ ID NO: 11 - protein, 554 aa, Thermomyces stellatus
APTATLDDRAAKTATVEIPSPEATVLGNVKNKVESFGGIPYAHPPTGDLRMRPPKRLTEPMGTFDGTGPAGACPQFVASNDAKHFLLDTLGTIANLPFVQNVTGQSEDCLSITVARPEGTKADAKLPVLYWIFGGGFELGWSSMYDGTDLINYGVEIGKPFIFVAVNYRVGGYGFMPGKELQDEGSSNAGLLDQRMGLEWVADNIAAFGGDPDKVTIWGESAGAISVFAQMALFDGDNTYKGKPLFRGGIMSSGSIVPTKPMDSDKAQAVFDKVVETGGCSDADDKLKCLRDLSYDDFLNAATSVPHLLSFNSLALSYLPRPDGRTLTDSPDQLVLNEKYAAVPMIIGDQEDEGTLFALFQSELTTKDKLVTYLKDYYFDDATEEQLSDLVDTYGSGLSAIREGSPHRTGLLNEIFPGFKRRAAILGDLVFTLSRRLFLLATSTLNKDVPSWSYLMSQNEGFPILGTFHGADIFQTFFGIKDNYAAKTVRGYFINFVYSQDPNGDGDTSFPEWPQWSDGNKLAHVFADRSELLDDDFREDSFNWLSENIDVLRF

SEQ ID NO: 12 - protein, 558 aa, Chaetomium globosum
APPEPPANVIEKRAAPTVTVSAGSIVGTTRLSTEAFNGIPFAKPPVGPLRLKPPVRHDSPLGVFDASGIAPACPQFLADSDSNEFLAKVIGTLTNLPFFQKALKISEDCLTVNVFRPKGTKAGDKLPVLFWIFGGGFELGWSSMYDGGPLVTNAMNMGKPYVFVAINYRVGGFGFMPGKEILADGSGNLGHLDQRMGLEWVADNIAAFGGDPDKVTIWGESAGAISVFNQMALYDGDNTYKGKPLFHGAIMNSGSIVPADPVDCPKGQAIYDQVVEKAGCASAKDTLACLREAPYDKFLAAANSVPAILSYNSVALSYLPRPDGKALTKSTDVLVQEGKYAAVPMIIGDQEDEGTLFALFQPNITTSARLVTYLKDLYFHRATNQQIEGLVDTYSPWIWDGSPFGSGILNEIYPGFKRLAALLGDIVFTLTRRIFLETATAVNPDVPAWSYIASYNQGTPIMGTFHGSDILQVFYGILPNYASKSIQSYYANFVYNQDPNNAAGGTSSKSKISEDWPRWTGKDRKLINFFANRNGYLKDDFREEAAAYLGSHIGALYF

SEQ ID NO: 13 - protein, 559 aa, Madurella mycetomatis
APPQPPAKVVEQRAAPTVTISTGTIIGSSRLATEAFNGIPFALPPVGPLRLKPPVRLNSTIGVFDGSGIAPACPQFLADTDSNELLASIIDLVTNLPFFQKALKISEDCLTINVIRPKGTKAGDKLPVLFWIFGGGFELGWTSMYDGGPLVTNAMQMGKPYIFVAVNYRVGGFGFMPGKEILKDGAANLGHLDQRMGLEWVADNIAAFGGDPDKVTIWGESAGAISVFNQMALYDGNNNYNGRPLFRAAIMNSGSIIPADPVDCPKGQEIYNAVVAKAGCASAMDTLACLRALPYEKFLSAANSVPSVLSYNSVALSYMPRPDGKVLTKSTDALVFEGKYAAVPMIIGSQEDEGTLFSLFQPNITTTNRLVSYLEDLFFHGASRQQIAGLVAKYPAQLWEGSPFRTGFLNEIYPGFKRMAAILGDLVFTLTRRIFLEHAVKANPAVPAWSYIASYNYGTPILGTFHGSDLLQVFYGILPNYASKSIQSFYANFVYNMDPNDSSGGTNSQSSKVKEKWPQWTGEQRTLIQFYADRNEYLEDDFRSEAAQYIAEHVNELHV

SEQ ID NO: 14 - protein, 557 aa, Sordaria macrospora
APAEPPTQVLDKRAAPTVTISTGTIVGANGILTEGFNGIPFALPPTGNLRLKPPVRLKSSLGVFDASGIAPACPQFLADTSSNEFLPKVIDKLVNTQLFNTALNVKEDCLTISVTRPKGTKAGDKLPVLFWIFGGGFELGWASMYDGAPLVTNAINMGKPYVYVAVNYRVGAFGFMPGKEILKDGASNLGHLDQRMGLQWVADNIAAFGGDPDKVTIWGESAGAISVFNQMSLYNGDNKYNGKPLFRGAIMNSGSIVPADPVDCPKGQKVYDTVVRNAGCSGAADTLACLRALPYDTFLKAANSVPAILSYTSVALSYLPRPDGKVLTDSADKLVVNKKYAAVPMIIGDQEDEGTLFSLFQSNITTTSKLVSYLNDVFFHDATESQIKSLVSTYSPLLSAGSPFGTGIFNEIYPGFKRLAAILGDLVFTLTRRVFLDAAASVNPSVPAWSYLASYDFGTPILGTFHGSDLLQVFYGILPNYASKSIQSYYANFVYNLDPNDASGGTSSKSKVAHNWPQWQKERKLVQFFANYAGYLKDDFRSDSYNWIKSNVDALYI

SEQ ID NO: 15 - protein, 558 aa, Podospora anserina
APPEPPTQVLEKRARPTVSIAQGNVVGVSRINTEAFNGIPFAQAPVGPLRLKPPVRTTASFGTYDAGGIAAACPQFLADTDSEDLLAKIIDTVVNTALFQKALKISEDCLNINVIRPAGTKAGDKLPVLFWIYGGGFELGWSSMYDGGPLVSNAMAAGKPYVFVAVNYRVGAFGFMPGKEVLQDGSANLGHLDQRMGLEWVADNIAAFGGDPDKVTIWGESAGAISVWNQMSLYDGNINYKGKPLFRGAIMNSGSIVPADPVDCPKGQEIYDTVVARSGCGGAADTLNCLRNLPYETFLEAANSVPAILSYSSVALSYLPRPDGVALTDSADRLVKAGKYAAVPMIIGDQEDEGTLFSLFQKNVTTTSRLEDYLRGYFFHGATAQQVKGLVSQYSPLISAGSPFGSGLFNEIYPGFKRLAALLGDIVFTLTRRVFIEYAIAANPNVPVWSYLASYNEGTPILGTFHGSDLLQVFYGILPNYASKSIQNFYANFVYNLNPNDASGGTSAKSKVKENWPTWNTKDKRLIQFFNNRNGYLKDDFREGAKQYIAANIDALHI

SEQ ID NO: 16 - protein, 557 aa, Neurospora tetrasperma
APAEPPTQVLDKRAAPTVTISTGMIVGANGILTEAFNGIPYALPPTGNLRLKPPVRLKSSLGVFDASGIGPACPQFLADTSSNEFLPQVIDKIVNTQLFKTILNVKEDCLTISITRPKGTKAGDKLPVLFWIFGGGFELGSASMYDGAPLVTNAINMGKPYVYVAVNYRVGGFGFMPGKEILKDGSSNLGHLDQRMGLQWVADNIAAFGGDPDKVTIWGESAGAMSVFNQMSLYDGDNTYNGKPLFRGAIMNSGSIVPAGPVDCPKGQKVYDTVVKNAGCSGAADTLACLRALPYETFLKAANSVPGILSYNSVALSYLPRPDGKVLTQSSDKLMLAKKYAAVPMIIGDQEDEGTLFSLFQSNITTTSKLVSYLNDIFFNDATESQIKSLVSTYSTLISAGSPFGTGLFNEIYPGFKRLAAILGDLIFTLSRRIFLDAATTLNPSVPAWSYLASYNFGTPILGTFHASDILQVFYGILPNYASKSIQSYYANFVYNLDPNDASGGTSSKSKVGQDWPQWQKERKLVQFFADYAGYLTDDFRSESYNWIKANIDALHI

SEQ ID NO: 17 - protein, 557 aa, Neurospora crassa
APAEPPTQVLHKRAAPTVTISTGTIVGANGILTEAFNGIPYALPPTGNLRLKPPVRLKSSLGVFDASGIGPACPQFLADTSSNEFLPQVIDKIVNTQLFKTILNVKEDCLTISVTRPKGTKAGDKLPVLFWIFGGGFELGSASMYDGAPLVTNAINMGKPYVYVAVNYRVGGFGFMPGKEILKDGSSNLGHLDQRMGLQWVADNIAAFGGDPDKVTIWGESAGAMSVFNQMSLYDGDNTYNGKPLFRGAIMNSGSIVPAGPVDCPKGQKVYDTVVKNAGCSGAADTLACLRALPYETFLKAANSVPGILSYNSVALSYLPRPDGKALTQSADKLMLAKKYAAVPMIIGDQEDEGTLFSLFQSNITTTSKLVSYLNDIFFNDATESQIKSLVSTYSTLISAGSPFGTGLFNEIYPGFKRLAAILGDLIFTLSRRIFLDAATTLNPSVPAWSYLASYNFGTPILGTFHASDILQVFYGILPNYASKSIQSYYANFVYNLDPNDASGGTSSKSKVSQDWPQWQKERKLVQFFSDYAGYLADDFRSDSYNWIKANLDALHI

SEQ ID NO: 18 - protein, 557 aa, Coniochaeta ligniaria
APTPNPVIEDRAAKVSVAIAPSSTVVGASTLGVEKFSGIPYAVPPVGPLRLKPPVRLTASLGTFDATGIEAACPQFLLSTGGNSLITQVLGNVLDLPFFQTVTKQSEDCLTINVIRPAGVKAGDNLPVLFWIFGGGFELGWTSMYDGSSLVSNAVNAGKPFVFVAVNYRVGGFGFLPGKEILQDGSANLGLLDQRMGLEWAADNIAAFGGDPSKVTIWGESAGSISVLDQMVLYGGNNQYNGKPLFRGAIMNSGSIVPADPVDCPKGQVVYDTVVRAAGCSSSTDTLACLRSADYTTFLNAANSVPGILSYNSVALSYLPRPDGTAITDSPDVLVLSGKYAAVPMIIGDQEDEGTLFSLFQSNTTTTSALIDYLNNIFFHDATREQIAALVATYDDSITAGSPFRTGIFNEVYPGFKRLAAILGDLVFQLTRRAFLTGHVAINPSVPAWSYMASYDYGTPILGSFHGSDLLQVFYGILPNAASKSIQSYYANFVYNLDPNDASGGTGTGTKVALGWPQWKDGNQLVQFFNIGSTLIKDDFRSGTFNWIVGNIPYLRI

SEQ ID NO: 19 - protein, 558 aa, Cutaneotrichosporon oleaginosum
APAPVVDDSHLDERALTITVSAPAGTIVGKRGLLTDDFNGIPYAAPPISNLRLRPPQRISSPLNNFDATKAAAACPQFLADSDDSGLLAQVMSTVSRTALFQRALKISEDCLSVNVIRPRGTKAGDNLPVLFWIYGGGFELGWSSMYDGGGLVQNAALNGKPYVFVAVNYRVGAWGFMPGKEILADGAGNLGLLDQRMGLEWVADNIAAFGGDPSRVTIWGESAGAISVWNQMTAYGGNIEYKGKPLFRGAIMNSGSIIPADPVDCPKGQAIYDAVVEKAGCSGPDSLACLRQLPLDQFTEAANSVPAILSYTSVALSYLPRPDGSFMPESPQQLLLKGRYAAVPMIIGDQEDEGTLFSLFQANTTETTPMIVRYLKDLFFNGASEATLTALVETYEDVPAEGSPFRTGPLNEVYKGFKRLAAILGDLVFTITRRIFLDIAKTVNPSVPAWSYIASYNYGTPILGTFHGSDLLQVFFGILPNKASRDIQAYYSNFVYNLDPNNYSGGTNRLSRVIDNWPQWERGTKKLIQFYAHHFQPMIDDFRTDSEQILSSNFESF

SEQ ID NO: 20 - protein, 556 aa, Sporothrix schenckii
APPSTPAIMERAAQVTMVLPLGTVIGTSSNGVESYNAIPFAQPPTGNLRLKPPVKRTASYGVLDGAGPAAACPQFVESTSGSLLSSVIGTLANSPFIQTTTDQTEDCLTIDVYRPAGTKATDKLPVLFWIFGGGFELGWTSMYDGAGLVAVSISLGKPMVFVAVNYRVGGFGFLPGAEVLADGASNLGLLDQRMGLEWTADNIGYFGGDSGKVTIWGESAGAISVADQMMLYNGNNTYKGKALFRGAIMDSGSIVPADPVDCPKGQAVYNQVVAKAGCSSSSDTLACLRAAPYNTFLNAVNSVPGILSYSSVALSYLPRPDGVVLVDSPDVLITNNKYAAVPMIIGDQEDEGTLFSLFQSNITNTNQLVTYLSDYYFHNATTAQIQALVATYPDNASAGSPFNTLLLNEVYPEFKRLAAILGDLVFTLTRRAFLAETNNRNPSVPSWSYLASYDYGTPVLGTFHGSDLLQVFYGILPNAASKSIHSYYFNFLYNQDPNNASGGTSSTASRVSLTWPQWGAGNQLVQFWAATSNLLADNFRNSSYTWITSNIASLHF

SEQ ID NO: 21 - protein, 549 aa, Stachybotrys chlorohalonata
LPKPDATLEERANSVTVALPSGTVIGASLLSVESFRGIPFADPPVGPLRLKPPQRLSEPLGTFDATGIAPACPQMFISTGTRDLIGGVLQELLDLPFLQPVTGQEDCLSITVQRPAGTQAGDKLPVLFWIFGGGFQLGSTLMYDGASLLATAVAQGQPFVFVAVNYRVAGFGFMPGAEILADGSANLGLLDQRMGLEWVADNIAAFGGDPDKVTIWGESAGSISVLDQMLLYGGDASYNGRPLFRGAIMNSGTVVPADPVDCPKGQEVYDTVVRNSGCEGSGDTLACLRELPYDTFLDAANSVPSLLSYSSVALSYLPRPDGTVLPDSPQTLVSQGRYHAVPMIVGDQEDEGTLFALFQPNLTTTAEMAAYLGDLFFHAPSVQQLTTMVQTWPVSLTAGSPFRTGLFNEWYPGFKRLAAILGDIVFTLTRRVFLESAAAANPHVPVWSYLSSYNYGTPVMGTFHGSDLLQVFYGILPNNAMRSCRTYYFNFLYNLDPNEGVGGYLRWPEWKEAKTLLWFQTAAKNSILRDDFRQESFNWISQNVNLLHI

SEQ ID NO: 22 - protein, 554 aa, Colletotrichum orchidophilum
VSDHVQPLEDRAVANATVVLQTATVIGNVMNNVESFGGIPYAKPPTGQLRLKPPVRLTGNIGTFDATGPAAACPQMLSSSDSENILFNLLGSIANLPFVQKATGQTEDCLTITVARPAGTKADAKLPVLYWIFGGGFELGWSSMYDGTGLVQHGVDISKPFVFVAVNYRVAGFGFMPGKEILADGSANLGLLDQRMGLEWVADNIAAFGGDPDKVTIWGESAGAISVFDQMALYNGDNKYNGKALFRGAIMNSGSIAPTDPVDCPKGQAVYDKVVAEAGCAGKADTLNCLREADYNTFLNAVTSVPGILSYNSLALSYLPRPDGKTLTASPDVLAKNGQYAAVPMIIGDQEDEGTLFGIFQPNLTTTAKLVTYLKDYYFATATEAQIEAYVATYDDGVTAVINGSPHRTGLLNEIFPGFKRRSAILGDLVFTLTRRVFLNLATAVQPGVPSWSYLSSYDYGTPILGTFHGSDLLQVFYGIKDNYAARSIRTYYTNFVYASDPNVGLNGAYPAWPQWSQGQTLLQFFADKASTLKDDFRKTSSDWILNNAGILYI

SEQ ID NO: 23 - protein, 554 aa, Colletotrichum incanum
VPDDFRPLEDRAASTATVVLDTATVVGNVMNKVESFGGIPYAKPPTGQLRLKPPVRLTENIGTFDATGPAAACPQMISSSESENILFDLLGDIANLPFVQKATGQTEDCLSITVARPQGTKADAKLPVLYWIFGGGFQLGWSSMYDGTSLVQHGVDIGKPFIFVAVNYRVAGFGFMPGKEILADGSANLGLLDQRMGLEWVADNIAAFGGDPDKVTIWGESAGAISVFDQMALYNGDNKYKGKALFRGAIMNSGSITPTDPVDCPKGQAVYDKVVSRAGCSGKADTLNCLREVDFTTFLNAVTSVPGILSYSSIALSYLPRPDGKTLTASPDVLAKNGQYAAVPMIIGDQEDEGTLFGVFQPNLTTTDKLVTYLKDYYFATATEAQIRAYVATYDDGLNAVINGSPHRTGLLNEIFPGFKRRSAVLGDLVFTLTRRVFLNLATAVNPSVPSWSYLASYNYGTPILGTFHGSDLLQVFYGVKDNYAARSIRTYYTNFVYALDPNVGSNGQYPNWPKWSQGQNLIQFFADKASTLKDDFRKTSSDWILNNAGSLYF

SEQ ID NO: 24 - protein, 554 aa, Colletotrichum tofieldiae
VPDDFQPLEDRAASTATVVLDTATVVGNVMNKVESFGGIPYAKPPTGQLRLKPPVRLTENIGTFDATGPAAACPQMISSSDSENILFDLLGDIANLPFVQKATGQTEDCLSITVARPQGTKADAKLPVLYWIFGGGFQLGWSSMYDGTSLVQHGVDISKPFVFVAVNYRVAGFGFMPGKEILADGSANLGLLDQRMGLEWVADNIAAFGGDPDKVTIWGESAGAISVFDQMALYNGNNKYKGKALFRGAIMNSGSITPTDPVDCPKGQAVYDKVVSRAGCSGKADTLNCLREVDYTTFLNAVTSVPGILSYSSIALSYLPRPDGKTLTASPDVLAKNGQYAAVPMIIGDQEDEGTLFGVFQPNLTTTDKLVSYLKDYYFATATEAQIRAYVATYDDGLNAVINGSPHRTGLLNEIFPGFKRRSAVLGDLVFTLTRRVFLNLATAVNPSVPSWSYLASYNYGTPILGTFHGSDLLQVFYGVKDNYAARSIRTYYTNFLYALDPNVGSNGQYPNWPKWSQGQNLIQFFADKASTLKDDFRKTSSDWILNNAGSLYF

SEQ ID NO: 25 - protein, 550 aa, Hypoxylon sp. EC38
LPAPTVILEKRATTTSVVIPQGTIIGSVSNGVENYGGIPFAQAPEGGLRLKPPQRLNTSLGTFDATGAGPSCPQMFFSTGGNDLLISVLGNLINTPLFQTVTDQKEDCLTMRVQRPAGTKSDAKLPVLFWIFGGGFELGSPQMYDASSLISNGVDQGKPFIFVSVNYRTGGFGFLGGKEVLADGASNLGLLDQRMGLEWVQDNIAAFGGDPEKVTIWGESAGAISVFDQMALYGGDNTYNGKKLFRAAIMNSGSVVPANPVDGIQAQNIYNTVVQEAGCASAADTLACLRSADYDTFLHAANSVPGILSYTSVALSYLPRPDGKVLPDSPDVLARNGQYAAVPMIIGDQEDEGTLFSLFQSNLTNTDELVDYIWKVFFPAVDRDDVVGLVNSYDPSITAGSPFNTGILNNIYPEFKRLAAILGDMVFTLTRRIFLQTTLSANPNVPSWSYLASYDYGTPILGTFHASDILQVFYGILPNYASKAIQSYYISFVYTMDPNDGTSSDFPQWPQWKDNRNLMQFWNNKSNLLADDFRKTAEEYISAHADIFLI

SEQ ID NO: 26 - protein, 546 aa, Aspergillus glaucus
APAILGAPTVTIPSPQATVAGQAGLKVESFNGIPFAQPPTGSLRLKPPQPIESALGDVDATGIAQSCPQFFFATDTSEFPGSVVGQLANIPLFQKITNAGEDCLTLNIRRPAGTTAEDKLPVLVWIFGGGFELGSSTTYDGASLVQNSIDLDMPIVFVAMNYRVGGFGFMPGAEILADGAANLGLLDQRLALEWVADNIAAFGGDPDKVTIWGESAGSISVFDQMMLYDGDIDYKGSPLFRGAIMNSGSVVPADPVDCDKGQNIYDTVVAYAGCDTAEDTLECLRGLDYTDFLNAANSVPGILSYNSVSLSYLPRPDGKVFTQSPDFAVQSGKYASVPFIVGDQEDEGTLFALFQSNLTTTDDLIEYFSDIFFFHISRDQLQALIDTYEDTAENGSPFRTGIFNNWYPQFKRLGAILGDLTFTLTRRLFLKYAKELKPDVPAWSYLASYDYGTPILGTFHGSDILQVFYGILPNYASRSIHSYYLSFVYELDPNKRRGEEFPEWPQWTNEEVELMQFFKDKGELLKDDFRNETYDFLLENTQAFRI

SEQ ID NO: 27 - protein, 549 aa, Eutypa lata
APAPTHVLEDRVVKTTVTTPQGTIVGSSVLGVESFAGIPFAEAPVGSLRLKPPQRLNSSLGDFDATGLGPSCPQMFFSTEGDLLSSVLGNLINTPLFQTVTGQTEDCLTMRVQRPAGTKADAKLPVLFWIFGGAFELGSAQMYDGSSLILNGVAQDKPFIFAAVNYRIGGFGFLPGKEVLADGAANLGLLDQRMGLEWVADNIAAFGGDPEKVTMWGESAGAISVFDQMALYGGDNKYNEKSLFRAAIMNSGSVVPADPVDCAKCQVVYDTVVEEANCASASDTLECLREADYDTFLNAANSVPGVLSYSSVALSYLPRPDGFALPNSPDLLAQAGQYAAVPMIIGDVEDEGTLFSLFQPNITTHDDIVDYLSELLFHGATREQLDALVSTYDPSIAAGSPFRTSFLNNVYPKFKTLAAILGDLVFTLTRRIFLRTTLAANPDVPAWSYLASYDYGTPILGTFHGSDLLQVFYGILPNYASAAFQSYYISFLYTMDPNEGTSDKFLEWPQWSENEDLMWIHNDDGELLADDFRAESCTFITDNVDALQI

SEQ ID NO: 28 - protein, 558 aa, Fusarium oxysporum
QDFPAPAQTIQERADKTATVTISSPDATILGKVANGVESFNGIPFAEAPTGQLRLRPPKRVENSLGEFDGTRLARACPQMLLSSENKNLLFEIAGDLSNHPFFQTVTGQSEDCLSITITRPEGTSQDAKLPVLFWIYGGGFQFGWSSMYDGTGLIKHGLDMRKPFIFVAINYRTSGFGFMPGKEVLADGASNLGLLDQRMALEWVADNIEAFGGDPSLVTLWGESAGAISILNQMALYDGDNTYNGKPLFRGAIMNSGSIVPADPVDCPKGQKVYDKVVKEAGCSGEEDTLNCLREVDYSTFLKAVTSVPAFLSYNSVALSYLPRPDGKVLTESPEILVGSKKYAAVPMIIGDQEDEGTLLAIFQPNLTTTEDLVRYLKDLHFHGASEAQLTELVKTYDEGISAVVNGSPFRTGPLNEVFPGFKRRAALLGDIAFTLSRRAFLTLANLANPTTPSWSYLATYNFGTPIFGTFHASDIIQVFFGILPNYASRSIRTYYINFLYDLDPNSGINGKYPNWPQWDQGQKLMNFEANRASLINDDFRKDSYEFLLQNAQSLYV

SEQ ID NO: 29 - protein, 554 aa, Fusarium avenaceum
SPNPDPSIDQRAAGTATVVLPSATVLGNVMNKVESFGGIPYAEAPEGKLRLRPPKRLQKSLGTFDATGPAGACPQMLSSTKSKDVLFQLLGSIANLPFVQTVTGQSEDCLTITVARPEGTKANAKLPVLFWIFGGGFQLGWSSMYDGTGLVNYGVGLNQPFIFVAVNYRVGGFGFLPGKEIQDEGSGNLGLLDQRMGLEWVADNIAAFGGDPDKVTIWGESAGAISVFDQMALYDGDHKYNGKPLFRGAIMNSGSVVPCEPLDGVKGQAVYDQVIETAGCVGKSDTLDCLRNLDYTDFLNAVNSVPGILSYNSLALSYLPRPDGRTLTASPDVLALNRKYAAVPMIIGNQEDEGTLFSIFQANLTSDEKLVTYLKDYYFHAATAPQLRDLVRTYGSGLGAITQNSPFRSGILNEIFPGFKKRAAILGDLVFILARRLFLQAATGVNGDVPSWAYMSSYDQGTPVLGTFHGSDLIQVFYGIKDNYAARSIRSYYINFVYSLDPNAASNAKYPSWPQWKNGHKLMQFFADKVTIINDDFRSASYDWLVKNIASLKF

SEQ ID NO: 30 - protein, 557 aa, Diaporthe ampelina
LPARQGSPVVNGRAASPTVVLSPSNTVVGLASGNVEKYAGIPFADPPTGSLRLKPPKKLSTNLGDAYDAINPAAACPQMLVSTGENQSLFLQVLGDLVSLPIIQAATNQSEDCLTVSVIRPAGVEAGANLPVLYWIFGGAFELGWAAMYDGTDLLDQAISQDQPYIFVAVNYRVAGFGFMPGKEILADGSSNLGLLDQRAGLEWVADNIEAFGGDPTKVTLWGESAGAISVFDQMALYGGDNNYNGKPLFRGAIMNSGSIVPADPVDCPKGQVIYDTVVKEGGCSGAADTLECLRALPYDKFLDAANSVPGFLGYQSVALSYLPRPDGTVLPDSPDVLAAQGRYAAVPMINGDQEDEGTIFTLFTSNVTTTQDLLEYLATYYFNSASDEQLTALLDTYPQDITQGSPFRTGILNEVYPQFKRISALLGDLVFTLSRRGFLENTNAANPSVPSWSYLASYNFGTPFLGTFHGSDLLQVFFGIYDNYAARSIRTYYFNFLHNLDPNVGYGSYPNWPQWKDTGEGNQLVQFYALSQNYLADDFRSDTYAFIKENIASLRI

SEQ ID NO: 31 - protein, 549 aa, Ophiostoma piceae
MPKRRDQSIEPRTTVNVNYPEGEVVGVSVLGIESFRGVPFAQPPVGNLRLKPPVRYTENIGTKDTTGIGPSCPQMYLSTGNGELLFQLVGNLINIPLFQTATLSSEDCLTLNIQRPAGTTSNSSLPVLFWIFGGGFELGTNQYYDGIDLLTEGISLGEPFIFVAINYRVGGFGFLGGKEIKADGSSNLGLLDQRIALEWVADNIASFGGDPSKVTIWGESAGSISVFDQMALYGGNNKYKGKALFRGGIMNSGSVVPAAPVDGVKAQAIYDHVVSEAGCAGTSDTLACLRTVDYTKFLTAVNSVPGIVSYSSIALSYLPRPDGVVLIDSPEEIVKNKQYAAVPMIIGDQEDEGTLFAVLPNNITSTAKIVQYFQDLYFYNATKEQLTAFVNTYPTDITAGSPFNTGIFNELYPGFKRLAAILGDMTFTLARRAFLQLCSEVNPDVPSWSYLASYDYGFPFLGTFHATDILQVFYGVLPNYASGSIQKYYINFVTTGDPNKGAAVDIQWPQWSAKKNILQIYATKAVIVADNFRAKSYEYLYNNIGIFRI

SEQ ID NO: 32 - protein, 528 aa, Pleurotus sapidus
TPLPRANSVTLDSATFTGTTSGRVTKFLGIPYAQPPTGDRRFRLPEPIPPYTGTVRATAFGPACPQQSARLPLPDGLASDVVDLIVNTAYKAVFPDNEDCLSINVVVPTSATPTSKLPVAVWIFGGGFELGSPSLYDGGLIVERSIQLGEPVIYVSMNYRLSAFGFLASQEVKDTGVGNLGLQDQREALRWIQKYISSFGGDPTKVTIWGESAGAISVALHMVANNGNHEGLFRGAFMQSGSPIPVGDISHGQTYYDAIAAETGCSSAADTLACLRSVPYATLKTAVDRTPFIFDYQSLALARIPRADGVFLTDNPQRLVQAGKVANVPFVTGDCDDEGTLFSLANLNVTTTSQVRTYIKTFFMPQSTNAELDQMLNHYPLDLTQGSPFDTGILNALSPQFKRLAAFQGDAVFQAPRRFFLQQRSGKQNTWAFLSKRFKVAPFLGSFHASDILNVYFGGELGDYLINFVNNLDPNGQGRGINWPKYTTSSPNLVTFNDNLLFPVTITQDTFRTDAINFLTGVTLANPL

(19) United States
(12) Patent Application Publication
(10) Pub. No.: US 2022/0162581 A1
(43) Pub. Date: May 26, 2022

(54) Solution Stable Enzyme Composition

(71) Applicant: AB Enzymes Oy, Rajamäki (FI)

(72) Inventors: Werner BESENMATTER, Darmstadt (DE); Katja PALMUNEN, Rajamäki (FI); Mirkka PERKKALAINEN, Rajamäki (FI); Leena LEHTIKARI, Rajamäki (FI)

(21) Appl. No.: 17/601,741

(22) PCT Filed: Apr. 2, 2020
(86) PCT No.: PCT/FI2020/050215

§ 371 Date: Oct. 6, 2021

(30) Foreign Application Priority Data
Apr. 8, 2019 (EP) 19167787.1

(51) Int. Cl. C12N 9/96; C12N 9/18; D21C 5/00; D21H 17/00

(52) U.S. Cl. CPC C12N 9/96; C12N 9/18; D21H 17/005; D21C 5/005; C12Y 301/01013

(57) ABSTRACT

A solution stable enzyme composition comprising an enzyme component, a stabilizer component, an optional antimicrobial preservative and water is disclosed, as well as its use in manufacturing of pulp and paper.

### FIELD OF THE INVENTION

The present invention relates to solution stable enzyme compositions that are suitable for use in industrial applications such as in the manufacture of pulp and paper.

### BACKGROUND OF THE INVENTION

Solution stable enzyme compositions intended for commercial use advantageously have sufficient stability during storage and satisfactory shelf life. Compositions comprising sterol esterase are useful for hydrolysis and synthesis of esters, for example esters of phytosterols, cholesterol and glycerol with carboxylic acids, particularly fatty acids. Such compositions can be applied industrially as processing aid, e.g. in the production and recycling of pulp and paper as far as the enzymes are active under conditions typically encountered in these processes.

To enhance the stability of aqueous solutions of enzymes and generally proteins, many strategies have been pursued, and reviewed by Gianfreda et al. in Molecular and Cellular Biochemistry 1991 pp 97-128. These strategies include protein modification by glycosylation, pegylation, cross-linking, site-directed mutagenesis and chemical modification of amino acids. Another strategy is the addition of excipients, for example pH buffers, salts, surfactants, amino acids, sugars, organic solvents, polymers and cyclodextrins.

For example hydroxypropyl-γ-cyclodextrin, sorbitol and the surfactant polyethylene glycol sorbitan monolaurate, also known under the trade name Tween 20, have been evaluated for their stabilizing ability on the protein porcine growth hormone by Charman et al. in Pharmaceutical Research 1993 pp 954-962, who concluded that in contrast to hydroxypropyl-γ-cyclodextrin and Tween 20, sorbitol was only marginally effective and offered no advantage against precipitation.

An excipient effective in stabilizing all protein and enzyme solutions is unknown. Nevertheless, particular excipients that stabilize specific enzymes have been found. For example serine protease inhibitors were found to stabilize lipases (EP2521732 B1). Lipolytic enzymes in liquid products for pulp and paper applications, for example Resinase®HT and Stickaway® from Novozymes disclosed in associated data sheets, are stabilized with propylene glycol. The pH of Stickaway® is between 6.5 and 8.5. At pH values above 8 the sterol esterase from Melanocarpus albomyces was clearly less stable, and more stable within the pH range between 3 and 7 in a setup by Kontkanen et al. in Applied Microbiology and Biotechnology 2006 pp 696-704.

Pleiss et al. in Journal of Molecular Catalysis B 2000 pp 491-508 corroborated that almost all lipases, many esterases and all known cutinases, just as serine proteases, have a catalytic triad composed of serine, histidine and aspartate.

Instead of aspartate, glutamate is present in sterol esterases' catalytic triad, which is composed of serine, histidine and glutamate.

A common lipase, also called triacylglycerol lipase and classified by the IUBMB Enzyme Nomenclature as EC 3.1.1.3, for example the lipases from Candida antarctica, Thermomyces lanuginosus and Rhizomucor miehei, investigated by Kontkanen et al. in Journal of Biochemistry 2004 pp 51-59, is highly active on glycerol esters, but is without measurable activity on sterol esters.

A sterol esterase, classified as EC 3.1.1.13, hydrolyzes many different esters, including the esters of sterol and glycerol with short and long chain carboxylic acids.

An acetylcholine esterase, classified as EC 3.1.1.7, hydrolyzes acetylcholine and other choline esters. The distinction of esterase and lipase due to their sequence and three-dimensional structure is reviewed by Fojan et al. in Biochimie 2000 pp 1033-1041.

Enzyme activities on plant and wood sterol ester mixtures were compared to activities on cholesterol esters by Kontkanen et al. in Journal of Biochemistry 2004 pp 51-59, who concluded that pure cholesterol esters can be used as model substrates for sterol esterases. Examples of sterols are cholesterol, phytosterols and ergosterol, which naturally occur in animals, plants and fungi, respectively. Sterol esterases from animal sources are genuine cholesterol esterases. Cholesterol esterases from microorganisms are in general terms sterol esterases. Anyhow, sterol esterases and cholesterol esterases are one enzyme class of EC 3.1.1.13.

To maintain stability of an aqueous solution of the sterol esterase from Melanocarpus albomyces, Kontkanen et al. in Enzyme and Microbial Technology 2006 pp 265-273 used 1% of the nonionic surfactant polyethylene glycol p-isooctyl-phenyl ether, also known under the trade name Triton X-100.

Thus, a stabilizing compound that is effective for all enzymes has not been reported and there is a need for providing liquid compositions where enzymes remain stable.

### SUMMARY OF THE INVENTION

Previously unanticipated, the inventors have found that certain organic polyhydroxy compounds stabilize an aqueous composition comprising an enzyme having WGESAG sequence motif and a catalytic triad composed of S, H and E/D. The stabilizing effect can be achieved even without the addition of a surfactant, if the organic polyhydroxy compound is present in an adequate concentration and has sufficient hydroxyl groups per molecule.

According to the first aspect of the invention is provided a solution stable enzyme composition comprising:
(a) an enzyme component comprising an enzyme characterized by having the amino acids sequence WGESAG and a catalytic triad composed of S, H and E/D;
(b) a stabilizer component comprising an organic sugar alcohol that has three or more hydroxyl groups per molecule, and wherein the stabilizer component comprises at least one fifth of the total weight of the enzyme composition;
(c) an optional antimicrobial preservative component preventing growth of a microorganism; and
(d) water having dissolved therein said components (a), (b) and (c);

wherein the enzyme component of the solution stable enzyme composition remains in solution for at least 4 weeks upon storage at 37° C.

According to another aspect of the invention is provided a solution stable enzyme composition comprising: (a) an enzyme component comprising an enzyme characterized by having the amino acids sequence WGESAG and a catalytic triad composed of S, H and E/D; (b) a stabilizer component comprising an organic polyhydroxy compound that has three or more, or four or more, hydroxyl groups per molecule; (c) an optional antimicrobial preservative component preventing growth of a microorganism; and (d) water having dissolved therein said components (a), (b) and (c).

In an embodiment the concentration of the stabilizer component in the composition is from 20 to 75% by weight, preferably from 25 to 70% by weight, more preferably from 30 to 65% by weight, and most preferably from 33 to 60% by weight. In an embodiment the lower limit of the concentration of the stabilizer component is 20% by weight, preferably 22%, 24%, 25% by weight, more preferably 26%, 28%, 30% by weight, most preferably 33% by weight. In an embodiment the upper limit of the concentration of the stabilizer component is 75% by weight, preferably 74%, 72%, 70% by weight, more preferably 68%, 66%, 65% by weight and most preferably 60% by weight.

In an embodiment the amount of water in the composition is at least 10% by weight, preferably at least 12%, 14%, 15% by weight, more preferably at least 16%, 18%, 20% by weight and most preferably at least 25% by weight.

In an embodiment the pH of the composition is between 2 and 10, preferably between 2.5 and 9, more preferably between 3 and 8 and most preferably between 3.5 and 7.5.

According to the second aspect of the invention is provided a use, and a method of using, of the solution stable enzyme composition according to the above aspects in the manufacture of pulp. In an embodiment the solution stable enzyme composition is used by adding it in such a way that it is brought into a contact with material used in the manufacture of pulp where enzymatic treatment is needed.

According to the third aspect of the invention is provided a use, and a method of using, of the solution stable enzyme composition according to the above aspects in the manufacture of paper. In an embodiment the solution stable enzyme composition is used by adding it in such a way that it is brought into a contact with material used in the manufacture of paper where enzymatic treatment is needed.

### SEQUENCE LISTINGS

The following sequences are mature protein sequences without signal peptides.

The sequences are written from amino- to carboxyl-terminus. The actual enzyme molecules present in an aqueous solution can be shortened from both endings of the sequences (amino- and/or carboxyl-terminus) without loss of enzyme function. For example, compared to SEQ ID NO: 1, Kontkanen et al. in Enzyme and Microbial Technology 2006 pp 265-273 found that the first 13 amino-terminal amino acids of the sequence SEQ ID NO: 1 are absent in their preparation of sterol esterase from Melanocarpus albomyces, since their sequence started with AAPXVEISTG. Thus, in an embodiment are disclosed N-terminally truncated enzymes having enzyme activity.

SEQ ID NO: 1 is the sequence of a sterol esterase from Melanocarpus albomyces.

SEQ ID NO: 2 is the sequence of a sterol esterase from Chaetomium thermophilum.

SEQ ID NO: 3 is the sequence of a synthetic enzyme derived from alignment of the sequences of the sterol esterases from Scytalidium thermophilum, Myceliophthora thermophila, Thielavia terestris, Corynascus thermophilus, Myriococcum thermophilum, Melanocarpus albomyces and Chaetomium thermophilum.

SEQ ID NO: 4 is the sequence of a sterol esterase from Myceliophthora thermophila.

SEQ ID NO: 5 is the sequence of a sterol esterase from Corynascus thermophilus.

SEQ ID NO: 6 is the sequence of a sterol esterase from Myriococcum thermophilum.

SEQ ID NO: 7 is the sequence of a sterol esterase from Thielavia australiensis.

SEQ ID NO: 8 is the sequence of a sterol esterase from Thielavia terestris.

SEQ ID NO: 9 is the sequence of a sterol esterase from Scytalidium thermophilum.

SEQ ID NO: 10 is the sequence of a sterol esterase from Malbranchea cinnamomea.

SEQ ID NO: 11 is the sequence of a sterol esterase from Thermomyces stellatus.

SEQ ID NO: 12 is the sequence of a sterol esterase from Chaetomium globosum.

SEQ ID NO: 13 is the sequence of a sterol esterase from Madurella mycetomatis.

SEQ ID NO: 14 is the sequence of a sterol esterase from Sordaria macrospore.

SEQ ID NO: 15 is the sequence of a sterol esterase from Podospora anserina.

SEQ ID NO: 16 is the sequence of a sterol esterase from Neurospora tetrasperma.

SEQ ID NO: 17 is the sequence of a sterol esterase from Neurospora crassa.

SEQ ID NO: 18 is the sequence of a sterol esterase from Coniochaeta ligniaria.

SEQ ID NO: 19 is the sequence of a sterol esterase from Cutaneotrichosporon oleaginosum.

SEQ ID NO: 20 is the sequence of a sterol esterase from Sporothrix schenckii.

SEQ ID NO: 21 is the sequence of a sterol esterase from Stachybotrys chlorohalonata.

SEQ ID NO: 22 is the sequence of a sterol esterase from Colletotrichum orchidophilum.

SEQ ID NO: 23 is the sequence of a sterol esterase from Colletotrichum incanum.

SEQ ID NO: 24 is the sequence of a sterol esterase from Colletotrichum tofieldiae.

SEQ ID NO: 25 is the sequence of a sterol esterase from Hypoxylon sp. EC38.

SEQ ID NO: 26 is the sequence of a sterol esterase from Aspergillus glaucus.

SEQ ID NO: 27 is the sequence of a sterol esterase from Eutypa lata.

SEQ ID NO: 28 is the sequence of a sterol esterase from Fusarium oxysporum.

SEQ ID NO: 29 is the sequence of a sterol esterase from Fusarium avenaceum.

SEQ ID NO: 30 is the sequence of a sterol esterase from Diaporthe ampelina.

SEQ ID NO: 31 is the sequence of a sterol esterase from Ophiostoma piceae.

SEQ ID NO: 32 is the sequence of a sterol esterase from Pleurotus sapidus CAH17527.

### DETAILED DESCRIPTION

Sterol esterases are enzymes that act on sterol ester bonds, particularly sterol esterases catalyze the hydrolysis, alcoholysis, acidolysis, transacylation, transesterification and/or synthesis of sterol esters. In an embodiment the sterol esterases of the invention belong to the class EC 3.1.1.13.

Enzymes are catalytic proteins.

Proteins are polypeptides.

Polypeptides are long chains of amino acids linked by amide bonds. In an embodiment peptides are molecules composed of up to 20 amino acids, and polypeptides are molecules composed of more than 20 amino acids.

The generally accepted IUPAC single letter abbreviations for amino acids and their side chains in polypeptides are utilized, particularly S for serine, H for histidine, A for alanine, G for glycine, E for glutamic acid and glutamate, D for aspartic acid and aspartate, W for tryptophan.

A fragment of an enzyme characterized by a specific amino acid sequence is a polypeptide having one or more amino acids absent from the amino- and/or carboxyl-terminus of said sequence, and/or one or more deletions and/or insertions of one or more amino acids in said sequence, for example due to alternative splicing, wherein the fragment has enzyme activity. In an embodiment a fragment of an enzyme has the same or similar enzyme activity, and optionally stability, as the non-fragmented enzyme.

Propylene glycol is 1,2-propanediol, which is an organic polyhydroxy compound with two hydroxyl groups per molecule. 1,2-propanediol exists in two enantiomers and mixtures thereof, including the racemic mixture, that are encompassed in this definition of propylene glycol.

Sugar alcohols are organic compounds that can be produced by hydrogenation of carbohydrates, particularly monosaccharides, disaccharides, trisaccharides, oligosaccharides and polysaccharides. The hydrogenation causes reduction of an aldehyde- or a ketone-group to a hydroxyl-group. Monosaccharides are aldehydes or ketones that have two or more hydroxyl-groups per molecule. Thus the sugar alcohols have three or more, or four or more, hydroxyl-groups per molecule.

Cyclitols are cycloalkanes that have a hydroxyl-group on three or more, or four or more, ring atoms. The cyclitols have three or more, or four or more, hydroxyl-groups per molecule.

Pulp is a wet mass of material that is originally obtained from plants. Such pulp is manufactured from e.g. wood, cotton, papyrus, straw, fruits, paper and rags.

The ester of cholesterol with linoleic acid is cholesteryl linoleate.

Thermophilic fungi are fungi that grow at 45° C., preferably at 50° C., or higher temperatures.

A thermostable enzyme is an enzyme that retains at least 50% enzyme activity after incubation in an aqueous composition or aqueous environment or aqueous solution at 50° C., preferably 60° C., more preferably 70° C., most preferably 75° C. for at least 5 minutes, preferably for at least 10 minutes, more preferably for at least 30 minutes, and most preferably for at least 1 hour. Preferably the thermostable enzyme retains at least 50% enzyme activity after incubation in an aqueous solution at 50° C. for at least 5 minutes. Enzyme activity can be determined according to Example 1 below.

The articles “a” and “an”, as well as “another”, are meant to refer to one or to more than one, that is to one or at least one, including several, of the grammatical object of “a”, “an” or “another”.

The word “comprise” and variations thereof such as “comprises” and “comprising” are meant inclusively and include additional possible components. These terms also may in certain embodiments include their narrow meaning “consisting of”.

In an embodiment of the enzymes according to the invention the catalytic triad is composed of serine, histidine and glutamate/aspartate, and said serine of the catalytic triad is embedded in the sequence WGESAG.

A catalytic triad is a complex of three amino acid residues involved in catalysis. Such residues of a catalytic triad function as nucleophile, base and acid during catalysis. Beside a catalytic triad, also an oxyanion hole and a hydrophobic substrate binding site is formed by active site residues of the enzyme. Residues of the catalytic triad can be identified by mutation experiments, by structure determination or by sequence alignment with homologues that have known catalytic residues. Examples of sequence alignment tools are Clustal Omega, PfamScan, Muscle and Emboss Needle, which uses the Needleman-Wunsch algorithm. These tools are available online, for example at https://www.ebi.ac.uk/services/all and at https://www.ebi.ac.uk/Tools/pfa/.

Catalytic triads can be found in hydrolase and transferase enzymes. The positions of the catalytic triad residues in SEQ ID NO: 1-9 are S221, H466 and E353. Examples for enzymes according to the invention include, but are not limited to enzymes comprising amino acid sequences disclosed in SEQ ID NO: 1-32 and fragments thereof. Further examples for enzymes according to the invention include, but are not limited to sterol esterases from Melanocarpus albomyces, Chaetomium thermophilum, Scytalidium thermophilum, Myceliophthora thermophila, Thielavia terestris, Thielavia australiensis, Corynascus thermophilus, Myriococcum thermophilum, Malbranchea cinnamomea, Thermomyces stellatus and other thermophilic fungi. Such thermophilic fungi include, but are not limited to thermophilic ascomycetes (e.g. Canariomyces thermophila, Chaetomidium pingtungium, Chaetomium britannicum, Chaetomium mesopotamicum, Chaetomium senegalensis, Chaetomium thermophile, Chaetomium virginicum, Corynascus sepedonium, Corynascus thermophilus, Crassicarpon thermophilum, Coonemeria aegyptiaca, Coonemeria crustacea, Dactylomyces thermophilus, Melanocarpus albomyces, Melanocarpus thermophilus, Myriococcum thermophilum, Crassicarpon hotsonii, Talaromyces byssochlamydioides, Talaromyces duponti, Talaromyces emersonii, Talaromyces thermophilus, Thermoascus aurantiacus, Thermomyces stellatus, Thielavia australiensis, Thielavia minor, Thielavia terricola), thermophilic zygomycetes (e.g. Rhizomucor miehei, Rhizomucor nainitalensis, Rhizomucor pusillus, Rhizopus microspores, Rhizopus rhizopodiformis) and thermophilic deuteromycetes (e.g. Acremonium alabamense, Acremonium thermophilum, Arthrinium pterospermum, Chrysosporium tropicum, Malbranchea cinnamomea, Myceliophthora fergusi, Myceliophthora hinnulea, Myceliophthora thermophila, Scytalidium indonesicum, Scytalidium thermophilum, Remersonia thermophila, Thermomyces ibadanensis, Thermomyces lanuginosus).

Genes and polypeptides derived from thermophilic microorganisms that express thermostable enzymes, are interesting for enzyme compositions stable during storage and application. Particularly genes derived from thermophilic fungi, that lead to high enzyme yields in fungal expression hosts, are particularly interesting for enzyme production and stability during storage and application.

Maheshwari et al. in Microbiology and Molecular Biology Reviews 2000 pp 461-488 reported that among the eukaryotic organisms, only a few species of fungi have the ability to thrive at temperatures between 45 and 55° C. Such fungi comprise thermophilic species, which are not as extreme as thermophilic species of bacteria and archaea. Maheshwari et al. estimated that only some 30 species out of approximately 50000 known fungal species breach this upper temperature limit of eukaryotes. Salar et al. in Journal of Agricultural Technology 2007 pp 77-107 reported 42 species of thermophilic fungi.

Further examples for enzymes according to the invention include, but are not limited to sterol esterases from fungi, particularly Basidiomycota, particularly Pleurotus species. Such examples for enzymes according to the invention also include, but are not limited to sterol esterases from Ascomycota, particularly Melanocarpus, Chaetomium, Chaetomidium, Corynascus, Crassicarpon, Canariomyces, Colletotrichum, Coonemeria, Crassicarpon, Dactylomyces, Malbranchea, Myriococcum, Neurospora, Ophiostoma, Talaromyces, Thermoascus, Thermomyces, Thielavia, Fusarium and Aspergillus species.

One method of demonstrating relationship among enzymes is sequence comparison.

Percentages of sequence identity were calculated with the algorithm Clustal Omega, available online at https://www.ebi.ac.uk/Tools/msa/.

TABLE 1
Percent identity matrix of closely related enzymes, sterol esterases
derived from thermophilic fungi

| SEQ ID | | | | | | | | | | |
|---|---|---|---|---|---|---|---|---|---|---|
| NO: | 1 | 2 | 3 | 4 | 5 | 6 | 7 | 8 | 9 | 10 |
| 1 | 100.0% | 80.7% | 82.8% | 84.6% | 85.7% | 84.6% | 81.0% | 79.2% | 75.5% | 63.0% |
| 2 | 80.7% | 100.0% | 97.9% | 78.1% | 78.7% | 77.6% | 75.8% | 74.6% | 75.8% | 60.2% |
| 3 | 82.8% | 97.9% | 100.0% | 80.3% | 80.8% | 79.8% | 78.0% | 76.7% | 78.0% | 61.7% |
| 4 | 84.6% | 78.1% | 80.3% | 100.0% | 91.8% | 91.6% | 80.8% | 79.2% | 76.5% | 61.9% |
| 5 | 85.5% | 78.7% | 80.8% | 91.8% | 100.0% | 91.2% | 79.4% | 77.8% | 75.5% | 62.0% |
| 6 | 84.6% | 77.6% | 79.8% | 91.6% | 91.2% | 100.0% | 79.9% | 78.3% | 76.9% | 63.3% |
| 7 | 81.0% | 75.8% | 78.0% | 80.8% | 79.4% | 79.9% | 100.0% | 77.2% | 74.4% | 60.6% |
| 8 | 79.2% | 74.6% | 76.7% | 79.2% | 77.8% | 78.3% | 77.2% | 100.0% | 71.9% | 61.3% |
| 9 | 75.5% | 75.8% | 78.0% | 76.5% | 75.5% | 76.9% | 74.4% | 71.9% | 100.0% | 60.4% |
| 10 | 63.0% | 60.2% | 61.7% | 61.9% | 62.0% | 63.3% | 60.6% | 61.3% | 60.4% | 100.0% |

TABLE 2
Percent identity matrix of enzymes distinct from sterol esterases

| | A | B | C | D | E | F | G | H | I | J | K |
|---|---|---|---|---|---|---|---|---|---|---|---|
| A: CAI96520 | 100% | 46% | 38% | 19% | 16% | 18% | 20% | 14% | 28% | 27% | 31% |
| B: P32947 | 46% | 100% | 42% | 15% | 14% | 18% | 18% | 15% | 26% | 27% | 28% |
| C: ACX69980 | 38% | 42% | 100% | 15% | 17% | 16% | 16% | 15% | 31% | 28% | 30% |
| D: CAA83122 | 19% | 15% | 15% | 100% | 6% | 16% | 20% | 23% | 13% | 14% | 14% |
| E: AAC08588 | 16% | 14% | 17% | 6% | 100% | 32% | 13% | 9% | 12% | 16% | 12% |
| F: CAA00250 | 18% | 18% | 16% | 16% | 32% | 100% | 11% | 16% | 16% | 18% | 16% |
| G: AMR67078 | 20% | 18% | 16% | 20% | 13% | 11% | 100% | 18% | 15% | 14% | 14% |
| H: P00590 | 14% | 15% | 15% | 23% | 9% | 16% | 18% | 100% | 13% | 11% | 11% |
| I: CAA36703 | 28% | 26% | 31% | 13% | 12% | 16% | 15% | 13% | 100% | 31% | 32% |
| J: P04058.2 | 27% | 27% | 28% | 14% | 16% | 18% | 14% | 11% | 31% | 100% | 32% |
| K: P37967.2 | 31% | 28% | 30% | 14% | 12% | 16% | 14% | 11% | 32% | 32% | 100% |

Caption to table 2: The used sequence accession codes stand for the following enzymes:

| | |
|---|---|
| A: CAI96520 | for the sterol esterase from Melanocarpus albomyces |
| B: P32947 | for the lipase 3 from Candida rugosa |
| C: ACX69980 | for the lipase from Geotrichum candidum |
| D: CAA83122 | for the lipase B from Candida antarctica |
| E: AAC08588 | for the lipase from Thermomyces lanuginosus |
| F: CAA00250 | for the lipase from Rhizomucor miehei |
| G: AMR67078 | for the lipase from Pseudomonas alcaligenes |
| H: P00590 | for the cutinase from Fusarium solani |
| I: CAA36703 | for the protein D2 from Dictyostelium discoideum |
| J: P04058.2 | for the acetylcholin esterase from Torpedo califomica |
| K: P37967.2 | for the para-nitrobenzyl esterase from Bacillus subtilis |

The serine of the catalytic triad of sterol esterases is embedded in the conserved sequence WGESAG. This sequence of WGESAG is absent in related but different enzymes with glutamate in the catalytic triad, e.g. acetylcholine esterase from Torpedo californica, para-nitrobenzyl esterase from Bacillus subtilis, lipases from Geotrichum candidum and Candida rugosa, which have instead the sequence FGESAG. Absence of the WGESAG sequence, actually of any GXSXG motif, in an unrelated family of cholesterol esterases derived from actinomycetes bacteria was revealed by Xiang et al. in Biochimica et Biophysica Acta 2007 pp 112-120. Different from common lipases, which are not active on sterol esters, the lipase from Candida rugosa is active on both substrates, glycerol esters and sterol esters. Beside substrate specificity, sequence features are determining properties to classify enzymes. In some cases sequence features are more accurate than specific activities as classification means. Due to the sequence-structure-function relationship, sequence features and substrate specificities are linked. Also other enzyme properties like stability and receptiveness to certain stabilizer components have their root cause in specific sequences.

Thus, in an embodiment the stabilizing effect the inventors have found for specific polyhydroxy compound is characteristic for the enzymes that preferably have both the WGESAG motif and the catalytic triad composed of S, H and E/D, wherein the S residue is part of said motif. In the Examples below, this stabilizing effect is shown for various enzymes carrying said motif and the catalytic triad, and with various polyhydroxy compounds. Thus, the above parameters define a limited set of enzymes and specific polyhydroxy compounds for which the stabilizing effect is shown.

An enzyme according to the invention can be isolated from its original biological source, or it can be produced in a cell-free system or produced as a secreted or intracellular protein in its original host or in an expression host, such as in a heterologous expression host. Such expression hosts are, but are not limited to filamentous fungi, yeasts, bacteria, plants and algae, for example Trichoderma reesei, Aspergillus oryzae, Pichia pastoris, Bacillus subtilis and Escherichia coli; and were reviewed by Fernandez et al. in Advanced Technologies for Protein Complex Production and Characterization 2016 pp 15-24, and by Yin et al. in Journal of Biotechnology 2007 335-347. Kontkanen et al. in Applied Microbiology and Biotechnology 2006 pp 696-704 described expression of the sterol esterase from Melanocarpus albomyces in Trichoderma reesei.

An enzyme according to the invention—and a gene encoding the enzyme according to the invention—can be derived from polypeptides and nucleic acids found in nature, or from a synthetic polypeptide or synthetic nucleic acid with sequence information derived from nucleic acids or polypeptides found in nature. Such sequence information can be derived from more than one sequence found in nature, for example calculation of a common ancestor sequence, calculation of a synthetic sequence from an alignment of known homologous sequences, particularly calculation of the most frequent amino acid at each position and derivation of a consensus sequence.

Furthermore an enzyme according to the invention—and a gene encoding the enzyme according to the invention—can contain one or more alterations without loss of function. Examples of such alterations are insertions, deletions and/or substitutions, preferably conservative substitutions, relative to a polypeptide or nucleic acid found in nature. The experimental exchangeability of amino acids in proteins was reviewed by Yampolsky and Stoltzfus in Genetics 2005 pp 1459-1472. Amino acid alterations are designated by their single letters separated by a slash, e.g. E/D means E or D. A particular example for such conservative substitutions are exchanges between E and D, because E and D are both acidic amino acids that differ only in one methylene spacer in their side chains. Further examples of the conservative substitutions are substitutions within the group of basic amino acids (e.g. R/K/H), acidic amino acids and their amides (e.g. E/D/N/Q), aromatic amino acids (e.g. W/F/Y), hydrophobic amino acids (e.g. F/L/I/V/A), tiny amino acids (e.g. G/A/S), medium amino acids (e.g. T/S/A/V/M/C), charged amino acids (e.g. E/D/R/K/H) and other polar amino acids (e.g. S/T/N/Q/H). Beside substitutions by the twenty canonical amino acids, also other genetically encoded amino acids, for example selenocysteine and pyrrolysine, and so-called unnatural amino acids can be incorporated in proteins. Example of such unnatural amino acids are reviewed by Wang et al. in Chemistry & Biology 2009 pp 323-336. By stepwise solid-phase peptide synthesis any available amino acid can be incorporated in peptides, which can be chemically ligated to larger peptides and polypeptides, to produce proteins and enzymes.

Furthermore an enzyme according to the invention can be a fusion polypeptide in which another polypeptide and/or oligopeptide is fused at the amino- and/or carboxyl-terminus to a polypeptide characterized by enzyme activity on sterol esters. Examples of such oligopeptides and polypeptides are, but are not limited to polyhistidine-tags, signal peptides, linkers, binding domains, antibodies, chaperones, fluorescent proteins and enzymes, for example with cholesterol oxidase activity.

The concentration of enzymes in compositions according to the invention is selected from 0.0001 to 10% by weight and any range inbetween. Such percentage is meant as active enzyme protein weight per total weight of composition. In another embodiment the enzyme concentration is from 0.01 to 10 by weight, preferable 0.1 to 8% by weight and more preferable 1 to 5% by weight. Such percentages are meant on dry matter basis. In another embodiment the enzyme concentration is specified as enzyme activity per composition weight and selected from 10 to 100000 SEU/g, preferable from 100 to 50000 SEU/g, more preferable from 200 to 30000 SEU/g and most preferable from 400 to 10000 SEU/g. The enzyme activity can be determined using the method described in Example 1 below.

In an embodiment the enzyme is a sterol esterase. Sterol esterases are versatile enzymes, which have broad substrate specificity. Beside sterol and glycerol esters of carboxylic acids (for example short and long chain carboxylic acids, saturated and unsaturated fatty acids), also other natural and artificial esters, for example polyesters (e.g. polyethylene terephthalate), polymers comprising vinyl acetate monomer (e.g. polyvinyl acetate) and para-nitrophenyl esters can be substrates and/or products of enzymes according to the invention. Therefore, such enzymes are useful in many industrial applications, particularly in pulp and paper, food, feed, textile, detergent, personal care and diagnostic industries. Specific examples within such industries are, but are not limited to use in a cleaning application, particularly for laundry and/or contact lenses, use as processing aid in manufacture of polyester textiles and/or wool, use in production and/or recycling of paper and/or pulp, use in a biosensor and/or a diagnostic reagent for measurement of total cholesterol (e.g. in blood) and use in synthesis of sterol esters. Examples of use of such sterol esters, particularly esters of cholesterol and phytosterol, more particularly stigmasteryl oleate, are use in food (e.g. added to margarines), feed, cosmetics and pharmaceutical formulations, as well as use in technical applications such as in liquid crystal display, which contains cholesterol esters.

Examples of sterols according to the invention include, but are not limited to, cholesterol, ergosterol, lanosterol, phytosterols, sitosterol, stigmasterol, campesterol, sitostanol, stigmastanol, campestanol, brassicasterol, fucosterol and cycloartenol.

Examples of sterol esters according to the invention include, but are not limited to, esters of cholesterol, esters of ergosterol, esters of lanosterol, esters of phytosterols, esters of sitosterol, esters of stigmasterol, esters of campesterol, esters of sitostanol, esters of stigmastanol, esters of campestanol esters of brassicasterol, esters of fucosterol and esters of cycloartenol.

Examples of esters of cholesterol according to the invention include, but are not limited to, cholesteryl linoleate, cholesteryl linolenate, cholesteryl myristoleate, cholesteryl palmitoleate, cholesteryl oleate, cholesteryl sapienate, cholesteryl arachidonate, cholesteryl erucate, cholesteryl crotonate. cholesteryl phenylpropionate, cholesteryl phenylacetate, cholesteryl cinnamate, cholesteryl benzoate, cholesteryl nitrobenzoate, cholesteryl dichlorobenzoate, cholesteryl chloroformate, cholesteryl formate, cholesteryl acetate, cholesteryl propionate, cholesteryl butyrate, cholesteryl valerate (cholesteryl pentanoate), cholesteryl caproate, cholesteryl heptanoate, cholesteryl octanoate (cholesteryl caprylate), cholesteryl nonanoate (cholesteryl pelargonate), cholesteryl decanoate (cholesteryl caprate), cholesteryl laurate, cholesteryl myristate, cholesteryl palmitate, cholesteryl stearate, cholesteryl eicosanoate (cholesteryl arachidate), cholesteryl docosanoate (cholesteryl behenate), cholesteryl tetracosanoate (cholesteryl lignocerate), cholesteryl hexacosanoate (cholesteryl cerotate), cholesteryl acetoacetate, cholesteryl hemisuccinate and cholesteryl isobutyrate.

Enzyme activity according to the invention can be measured using a sterol ester as enzyme substrate and measuring pH change or applying a pH-stat method, which measurers the release of carboxylic acid from the corresponding ester, or the method described below in Example 1, which measures the release of cholesterol from cholesteryl linoleate. In this detailed described method cholesteryl linoleate can be replaced by other esters of sterol, particularly esters of cholesterol, more particularly esters of cholesterol as exemplified above.

In an embodiment the enzyme component is spent fermentation broth containing the enzyme. The spent fermentation broth is obtainable e.g. by recombinant production of the enzyme. The spent fermentation broth may be concentrated and/clarified after production of the enzyme. In another embodiment a mixture of spent fermentation broths from several fermentations can be used.

In an embodiment the solution stable enzyme composition, and the enzyme component, remains in solution upon storage at 37° C. for at least 4 weeks, preferably for at least 24 weeks. When the enzyme remains in solution, it does not develop turbidity or precipitation due to the enzyme. A solution stable enzyme composition that can keep an enzyme in solution at 37° C. for at least 4 weeks, preferably at least 24 weeks, implies that the enzyme remains soluble and catalytically functional. If an enzyme loses its catalytic property, then it isn't an enzyme anymore, but for example a denatured protein.

Growth of microorganisms or other reasons may cause development of turbidity or precipitation during storage at 37° C. In conditions that are potentially suitable for microbial growth it may be preferable to include an antimicrobial preservative in the composition.

In an embodiment the solution stable enzyme composition remains clear after 4 weeks storage at 4° C., preferably after 8 weeks storage at 4° C., more preferably after 24 week storage at 4° C. In an embodiment the solution stable enzyme composition remains clear after 4 weeks storage at 20° C., preferably after 8 weeks storage at 20° C., more preferably after 24 week storage at 20° C. In an embodiment the solution stable enzyme composition remains clear after 4 weeks storage at 37° C., preferably after 8 weeks storage at 37° C., more preferably after 24 week storage at 37° C. Thus, a solution stable enzyme composition is a composition which does not significantly turn turbid or precipitate during storage.

In an embodiment the solution stable enzyme composition has after 4 weeks storage at 4° C., preferably after 8 weeks storage at 4° C., a remaining enzyme activity of more than one third, preferably more than half of its activity compared to the enzyme activity directly after preparing such enzyme composition. In an embodiment the solution stable enzyme composition has after 4 weeks storage at 20° C., preferably after 8 weeks storage at 20° C., a remaining enzyme activity of more than one third, preferably more than half of its activity compared to the enzyme activity directly after preparing such enzyme composition. In an embodiment the solution stable enzyme composition has after 4 weeks storage at 37° C., preferably after 8 weeks storage at 37° C., a remaining enzyme activity of more than one third, preferably more than half of its activity compared to the enzyme activity directly after preparing such enzyme composition.

A polyhydroxy compound according to the invention is an organic polyhydroxy compound that has three or more, or four or more, hydroxyl groups, preferably four or more hydroxyl groups, more preferably five or more hydroxyl groups, per molecule. Examples of organic polyhydroxy compounds include, but are not limited to, pentaerythritol, trimethylolpropane, polyvinyl alcohol, certain carbohydrates, cyclitols and sugar alcohols. Examples of sugar alcohols according to the invention include, but are not limited to, sorbitol, mannitol, xylitol, glycerol, erythritol, threitol, arabitol, ribitol, galactitol, fucitol, iditol, inositol, volemitol, isomalt, maltitol, lactitol, maltotriitol, maltotetraitol, polyglycitol and hydrogenated starch hydrolysates. Inositol is an example for a sugar alcohol and simultaneously for a cyclitol. Naturally occurring cyclitols have six ring atoms and four or more hydroxyl-groups on ring atoms. Examples of such cyclitols are inositol, bornesitol, conduritol, ononitol, pinitol, pinpollitol, quebrachitol, valienol, viscumitol, ciceritol.

In an embodiment the stabilizer component comprises an organic polyhydroxy compound that has four or more hydroxyl groups, or five or more hydroxyl groups, per molecule. In a preferred embodiment the stabilizer component comprises a sugar alcohol which has four or more hydroxyl groups per molecule.

In an embodiment the stabilizer component does not comprise sugar.

In an embodiment the stabilizer component does not comprise monosaccharides.

In an embodiment the stabilizer component does not comprise polysaccharides.

In an embodiment the stabilizer component does not comprise saccharides.

In an embodiment the stabilizer component does not comprise starch.

In an embodiment the organic polyhydroxy compound consists of the elements carbon, hydrogen and oxygen only. In another embodiment the organic polyhydroxy compound consists of the elements carbon, hydrogen, oxygen and another element. Examples of such element are nitrogen, sulfur, phosphorus, boron, fluorine, chlorine, bromine and iodine.

In an embodiment the stabilizer component comprises a synthetic organic polyhydroxy compound which is preferably added in the composition. Thus, preferably the stabilizer component is not naturally present in the composition in a significant amount.

In an embodiment the stabilizer component comprises a mixture of sorbitol and glycerol.

In another embodiment the stabilizer component comprises a mixture of maltitol and sorbitol; a mixture of maltitol and glycerol; or a mixture of sorbitol and maltitol and glycerol. In another embodiment the stabilizer component comprises a mixture of mannitol and sorbitol; a mixture of mannitol and glycerol; or a mixture of sorbitol and mannitol and glycerol. In another embodiment the stabilizer component comprises a mixture of xylitol and sorbitol; a mixture of xylitol and glycerol; or a mixture of sorbitol and xylitol and glycerol.

In an embodiment the stabilizer component comprises a mixture of two or more sugar alcohols selected from the group consisting of sorbitol, glycerol, maltitol, mannitol and xylitol in any combination.

In an embodiment a mixture of sugar alcohols is used instead of a single sugar alcohol. Any appropriate mixture of the sugar alcohol can be used unless the sugar alcohols are incompatible with each other, with the enzyme component, or any other component present in the enzyme composition.

An upper limit for the concentration of sugar alcohols in compositions according to the invention is their limit of solubility. Aqueous sorbitol solutions are commercially available at a concentration of 70% by weight and maltitol solutions at a concentration of 75% by weight. A lower limit for the concentration of sugar alcohols in compositions according to the invention is their effectiveness as stabilizing excipient in compositions according to the invention. The effectiveness can be determined using the method described in Example 2 below.

In an embodiment the solution stable enzyme composition contains by weight at least 20%, 24%, 25%, 28%, 30%, 33%, 35%, 40% or 50% of said sugar alcohol. Such embodiments with high concentrations of said sugar alcohol are advantageous because such compositions remain liquid at temperatures below 0° C. Such compositions can be stored at temperatures below 0° C., for example stored outside in winter, without freezing. The freezing points of compositions with high concentrations of sorbitol, maltitol, lactitol, and hydrogenated corn syrup were reported by Uraji et al. in Food Science Technology International 1996 pp 38-42.

In an embodiment the solution stable enzyme composition contains a mixture of at least two different sugar alcohols. Various ratios of different sugar alcohols are efficient in providing storage stability, as shown in the examples below. The first sugar alcohol may be present for example at about 20, 25, 30, 40, or 50% by weight, whereas the second sugar alcohol may be present for example at about 5, 6, 7, 8, 9, 10, 15, 20, 25 or 30% by weight.

Antimicrobial preservation means are especially important during long storage at ambient temperatures of aqueous compositions comprising organic compounds, especially if such compositions have not been sterilized. Sterilization of liquid products is less common if such products are used for technical applications rather than for medical applications. Thus, in the present invention the solution stable enzyme composition preferably contains antimicrobial preservative to prevent microbial growth.

Antimicrobial preservatives according to the invention are preferably antibacterial chemical compounds and/or antifungal chemical compounds, more preferably chemical compounds against molds, yeasts and/or acid-tolerant bacteria. Such antimicrobial preservatives are for example fungicides, fungistatics, bactericides and/or bacteriostatics. Examples of antimicrobial preservatives according to the invention include, but are not limited to isothiazolinones, particularly 1,2-benzisothiazolin-3-one, 2-methyl-4-isothiazolin-3-one, 5-chloro-2-methyl-4-isothiazolin-3-one, 2-octylisothiazolin-3-one and 4,5-dichlor-2-octylisothiazolin-3-one, phenoxyethanol, benzoic acid, hydroxybenzoic acids, particularly 4-hydroxybenzoic acid and 2-hydroxybenzoic acid, which is also called salicylic acid, sorbic acid, propionic acid, lactic acid, hexanoic acid, octanoic acid, sulfur dioxide, furthermore salts of these acids, particularly sodium, potassium and calcium benzoate, hydroxybenzoate, salicylate, sorbate, propionate, hexanoate, octanoate, sulfite, bisulfite and metabisulfite. Further examples of antimicrobial preservatives are esters of hydroxybenzoic acids, for example methyl 4-hydroxybenzoate, ethyl 4-hydroxybenzoate, propyl 4-hydroxybenzoate, butyl 4-hydroxybenzoate, heptyl 4-hydroxybenzoate, isobutyl 4-hydroxybenzoate, isopropyl 4-hydroxybenzoate, phenyl 4-hydroxybenzoate and benzyl 4-hydroxybenzoate (which are also called methylparaben, ethylparaben, propylparaben, butylparaben, heptylparaben, isobutylparaben, isopropylparaben, phenylparaben and benzylparaben respectively) as well as corresponding salts thereof, for example sodium methyl 4-hydroxybenzoate, sodium ethyl 4-hydroxybenzoate, sodium propyl 4-hydroxybenzoate and so forth.

In a further embodiment instead of, or in addition to, the antimicrobial preservative component, a high concentration of the stabilizer component is used to prevent microbial growth. A sufficiently high concentration is a concentration, which entails an antimicrobial effect due to high osmotic pressure and/or low water activity. Thus, the stabilizer component according to the invention may function simultaneously as antimicrobial preservative according to the invention. Examples of such compounds at high concentrations are sucrose at concentrations of at least approximately 50% by weight, sorbitol at concentrations of at least approximately 45% by weight, glycerin at concentrations of at least approximately 25% by weight and mixtures of glycerin and sorbitol at various high concentrations as determined by Barr and Tice in Journal of the American Pharmaceutical Association 1957 pp 217-223.

Compounds having antimicrobial preservative effect are used according to the invention at concentrations from 0.002 to 75% by weight, preferably from 0.005 to 10% by weight, more preferably from 0.01% to 5% by weight, even more preferably from 0.02 to 2% by weight, most preferably from 0.04 to 0.5% by weight. A lower limit for the concentration of antimicrobial preservatives in compositions according to the invention is their effectiveness to prevent growth of a microorganism in such compositions.

An antimicrobial effect can be determined by counting viable cells (for example as colony forming units) in an aqueous composition (for example a composition according to the invention) after inoculation with a microorganism (for example the acid-tolerant bacterium Lactobacillus buchneri, the mold Aspergillus oryzae or the yeast Pichia pastoris) and incubation for one or more, particularly several weeks at a storage temperature (for example 8° C., 25° C. or 37° C.) and comparing with a similar aqueous composition without antimicrobial preservative and/or comparing with inoculated aqueous composition before incubation.

Acid-tolerant bacteria, molds and yeasts are food spoilage microorganisms.

To achieve long storage stability of compositions according to the invention, the addition of protease inhibitors, antioxidants or surfactants was not necessary.

The present invention is further described by referring to the following embodiments.

In an embodiment the solution stable enzyme composition is for industrial use. In another embodiment it is for use in manufacturing of paper and/or pulp.

In an embodiment the pH of the solution stable enzyme composition is selected in the range from strongly acidic over neutral to slightly alkaline. In an embodiment the pH of the solution stable enzyme composition is selected in the acidic range. In an embodiment the pH of the solution stable enzyme composition is selected in the citrate buffered range, which is from pH 3.0 to 6.2. In an embodiment the pH of the solution stable enzyme composition is selected in the acetate buffered range, which is from pH 3.5 to 5.8. In an embodiment the pH of the solution stable enzyme composition is selected in the phosphate buffered range, which is from pH 5.8 to 8.0. In an embodiment the pH of the solution stable enzyme composition is selected in an unbuffered neutral range. In an embodiment pH of the solution stable enzyme composition is buffered to a pH range 3.7-8.1 with a pH buffer solution, preferably citrate buffer, acetate buffer or phosphate buffer. In an embodiment the pH of the solution stable enzyme composition is selected from the range between 3 and 8. These ranges are particularly advantageous because within said ranges a good stability can be achieved for a long time with the specific polyhydroxy compounds of the invention, as is revealed in the examples. Furthermore within said ranges a good stability can be achieved with buffer substances that are compatible during a long storage time with all components of the composition according to the invention.

In an embodiment the enzyme is a hydrolase characterized by having enzyme activity on a sterol ester, preferably an ester of sterol with a fatty acid, more preferably an ester of cholesterol with linoleic acid.

In an embodiment the solution stable enzyme composition comprises fermentation broth, or clarified and optionally concentrated fermentation broth.

In an embodiment said enzyme is a thermostable enzyme. Said embodiment is advantageous because thermostability correlates with other types of stability, particularly long-term stability at ambient temperatures. Furthermore, said embodiment is also advantageous for use in the manufacturing of pulp and paper, where temperatures above 50° C., preferably above 60° C., more preferably above 70° C., most preferably above 75° C., are frequently applied. In an embodiment said use in the manufacturing of pulp and paper is at an acidic pH, preferably at pH 5.0. In another embodiment said use in the manufacturing of pulp and paper is at a pH from 3.0 to 8.0.

In an embodiment said enzyme is a sterol esterase derived from a fungus, preferably a thermophilic fungus.

In an embodiment said enzyme is a fungal sterol esterase, preferably a thermophilically fungal sterol esterase.

In an embodiment said enzyme is a sterol esterase and the catalytic triad is composed of S, H and E.

In an embodiment said enzyme is a sterol esterase derived from a thermophilic fungus selected from the group consisting of Scytalidium thermophilum, Myceliophthora thermophila, Thielavia terestris, Corynascus thermophilus, Myriococcum thermophilum, Thermomyces stellatus, Thielavia australiensis, Malbranchea cinnamomea, Melanocarpus albomyces and Chaetomium thermophilum and fragments and conservative alterations of such sterol esterases. In another embodiment the enzyme is the closest sterol esterase homolog, in the above group of thermophilic fungi, of the enzyme having the amino acid sequence according to any one of SEQ ID NO: 1-32.

In an embodiment said enzyme is a sterol esterase encoded by a gene from the genome of a thermophilic fungus selected from the group consisting of Scytalidium thermophilum, Myceliophthora thermophila, Thielavia terestris, Corynascus thermophilus, Myriococcum thermophilum, Thermomyces stellatus, Thielavia australiensis, Malbranchea cinnamomea, Melanocarpus albomyces and Chaetomium thermophilum and fragments thereof and conservative alterations thereof.

In an embodiment said enzyme has at least 60%, preferably at least 70%, more preferably at least 75%, most preferably at least 80% sequence identity with a sterol esterase from Melanocarpus albomyces or Chaetomium thermophilum.

In an embodiment said enzyme has at least 60%, preferably at least 70%, more preferably at least 75%, most preferably at least 80% sequence identity with a sterol esterase from SEQ ID NO: 1 or SEQ ID NO: 2.

In another embodiment the enzyme has at least 50, 60, 70, 75, 80, 85, 90, 95 or 99% sequence identity with the corresponding amino acid with SEQ ID NO: 1 or 2.

In another embodiment the enzyme has at least 50, 60, 70, 75, 80, 85, 90, 95 or 99% sequence identity with the corresponding sequence of SEQ ID NO: 1, 2, 3, 4, 5, 6, 7, 8, 9 or 10, and preferably being a thermostable sterol esterase, more preferably a fungal thermostable sterol esterase.

In another embodiment the enzyme has at least 50, 60, 70, 75, 80, 85, 90, 95 or 99% sequence identity with the corresponding sequence of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31 or 32.

In an embodiment the enzyme does not have 100% sequence identity with any of the sequences SEQ ID NO: 1-32.

In an embodiment said polyhydroxy compound has three or more, preferably four or more, more preferably six or more hydroxyl groups per molecule.

In an embodiment said polyhydroxy compound has four or more, preferably five or more, more preferably six or more hydroxyl groups per molecule. Said polyhydroxy compounds are particularly advantageous in being able to stabilise various enzymes of the invention, because the stabilizing effect increases with increasing concentration of said polyhydroxy compound and with increasing number of hydroxyl groups. More hydroxyl groups per molecule causes higher molecular mass and improved water associated properties and water protein interaction.

In an embodiment said polyhydroxy compound is a sugar alcohol.

In an embodiment said sugar alcohol is selected from the group consisting of sorbitol, mannitol, maltitol, xylitol and glycerol.

In an embodiment said sugar alcohol is selected from the group consisting of sorbitol, mannitol, maltitol, and xylitol. Said embodiment is advantageous in applications where presence of glycerol is not desirable, such as in applications where glycerol interferes with function of enzymes, or is not compatible in the use. Because glycerol is a product of hydrolysis of glycerol esters, glycerol may cause decrease of enzyme activity due to product inhibition. Thus, instead of glycerol, usage of a sugar alcohol that has four or more hydroxyl groups per molecule, preferably from the group consisting of sorbitol, mannitol, maltitol and xylitol, may be desirable when applying a composition according to the invention for hydrolysis of glycerol esters. An example for an industrial application of enzymatic hydrolysis of glycerol esters is in the manufacturing of pulp and paper. Glycerol esters and sterol esters are present in wood resin, which causes sticky deposits during manufacturing of wood pulp and paper, and are preferably hydrolysed quickly before deposition occurs.

In an embodiment the stabilizer component constitute by weight at least one fifth, preferably at least one fourth, more preferably at least one third of said enzyme composition. These amounts are preferable because they provide the stabilizing effect for a long time.

In an embodiment the stabilizer component comprises by weight at least one fifth of the total weight of the enzyme component, preferably at least one fourth, more preferably at least one third. The stabilizing effect increases with increasing concentration, depending on the specific stabilizer component.

In an embodiment the solution stable enzyme composition comprises antimicrobial preservative selected from isothiazolinones, preferably selected from the group consisting of 1,2benzisothiazolin-3-one, 2-methyl-4-isothiazolin-3-one, 5-chloro-2-methyl-4-isothiazolin-3-one, 2-octylisothiazolin-3-one and 4,5-dichlor-2-octylisothiazolin-3-one. These preservatives are preferable because they are compatible with enzyme compositions and provide antimicrobial effect for a long time.

In an embodiment said preservative is isothiazolinone, preferably selected from the group consisting of 1,2-benzisothiazolin-3-one, 2-methyl-4-isothiazolin-3-one, 5-chloro-2-methyl-4-isothiazolin-3-one, 2-octylisothiazolin-3-one and 4,5-dichlor-2-octyl isothiazol in-3-one.

In an embodiment said preservative is selected from the group consisting of benzoic acid, 4-hydroxy-benzoic acid, 2-hydroxy-benzoic acid, and salts thereof and esters thereof. Such an embodiment containing preservatives that are allowed to be added to food are advantageous as a necessary prerequisite for food grade registration of compositions according to the invention. Food and food contact approval may also be required or advantageous for use in the manufacturing of food, feed, pulp and paper.

In an embodiment said preservative is active against at least one microorganism selected from the group consisting of acid-tolerant bacteria, molds and yeasts.

In an embodiment the solution stable enzyme composition is essentially free from polyethylene glycol p-alkyl-phenyl ethers, preferably free from all nonionic surfactants, more preferably free from all surfactants. This embodiment is particularly useful in applications where presence of surfactants is not desired, for example undesirable foam formation due to surfactants.

In an embodiment the solution stable enzyme composition is essentially free from serine protease inhibitors, preferably free from all protease inhibitors. Said embodiment is advantageous when simultaneous activity of a protease and an enzyme composition according to the invention is applied, for example in detergent compositions, for washing laundry and other cleaning applications.

The present invention is further described by the following examples, which do not limit the scope of the invention, which is defined by the appended claims and their equivalents.

### EXAMPLES

### Example 1—Measurement of Hydrolytic Enzyme Activity on the Sterol Ester Cholesteryl Linoleate

This method is a modification of the methods described by Stepien et al. in Acta Biochimica Polonica 2013 pp 401-403 and by Sigma-Aldrich at http://www.sigmaaldrich.com/technical-documents/protocols/biology/assay-procedure-for-cholesterol-esterase.html. The following chemicals were purchased from Sigma-Aldrich (now Merck) with the associated order numbers: cholesteryl linoleate CO289, cholesterol oxidase C8649, peroxidase from horseradish 77332, lyophilized bovine serum albumin A2153, 4-aminoantipyrine A4382, sodium 3,5-dichloro-2-hydroxybenzenesulfonate D4645, Triton X-100 X100. The following solutions were freshly prepared:

The AA-solution was 1.76 g 4-aminoantipyrine dissolved in 100 ml water. The DHBS-solution was 6 g sodium 3,5-dichloro-2-hydroxybenzenesulfonate dissolved in 100 ml water. The CL-solution was 9.8 mg cholesteryl linoleate dissolved in 0.5 ml isopropanol and a 1% Triton X-100 solution at 75° C. added, cooled to 25° C. and filled up to 25 ml with 1% Triton X-100. The peroxidase powder was dissolved and diluted to 150 PU/ml with 0.1M potassium hydrogenphosphate buffer pH 7.0. The cholesterol oxidase powder was dissolved and diluted to 30.3 U/ml with ice-cold water. The buffer solution, which was used for sample dilutions, was 0.095 g magnesium chloride, 0.0695 g sodium salt of ethylenediaminetetraacetic acid and 1 g lyophilized bovine serum albumin dissolved in 500 ml 0.2M potassium hydrogenphosphate buffer pH 7.5. The substrate solution was a mixture of 4.36 ml 0.2M potassium hydrogenphosphate buffer pH 7.0, 2.91 ml CL-solution, 0.145 ml AA-solution, 0.291 ml DHBS-solution and 0.291 ml peroxidase solution. From this substrate solution 0.917 ml were pipetted into a cuvette and incubated at 37° C. Then 0.033 ml cholesterol oxidase solution was added. Then 0.033 ml of the sample to be measured was quickly mixed into the cuvette. Immediately afterwards the change of absorption per minute, abbreviated ΔOD/min, was measured in the spectrophotometer Lambda25 from Perkin Elmer at 37° C. and 512 nm. The enzyme activity, sterol esterase units, abbreviated SEU, was calculated with the formula: SEU/ml=ΔOD/min—3.819, which takes into account the sample volume of 0.033 ml in a total volume of 0.983 ml and a light path through the cuvette of 1 cm. If the sample had been diluted before adding it into the cuvette, the result was also multiplied with the appropriate dilution factor. Samples were diluted in order to measure ΔOD/min values between 0.06 and 0.22.

Significant enzyme activities were measured with the method described above in samples prepared through expression in Trichoderma reesei of SEQ ID: 1, 2, 3, 5, 6, 9 and 10. Expression in Trichoderma reesei was done as known in the art, for example through Kontkanen et al. in Applied Microbiology and Biotechnology 2006 pp 696-704.

### Example 2—Experimental Evaluation of Liquid Enzyme Composition Stabilities

In all experiments enzyme materials used for preparing test compositions were either clarified fermentation broths or concentrates of them, containing preservative to prevent microbial growth, from several different fermentations of enzyme proteins. Studied enzyme proteins were sterol esterase from Melanocarpus albomyces, sterol esterase from Chaetomium thermophilum and enzyme with the synthetic sequence of SEQ ID NO: 3, which were expressed in Trichoderma reesei. In the test compositions enzyme liquids were standardized to activity level range from 1200 to 1900 SEU/g and stabilized using different stabilizing conditions as shown in the following tables. All test compositions were taken under accelerated storage stability study. Total storage time was 24 weeks. For stability study each liquid was divided in 15 ml screw cap tubes, 5 ml of liquid per tube. One tube was prepared for each storage time point. Starting point sample tubes were placed to freezer right after preparing and visual observation. All other sample tubes were placed to 37° C. climate chamber. From each time point one sample tube was taken under visual observation and placed to freezer before activity analysis. Liquid appearance from each storage time point were compared to the appearance at start. Also activities from different storage time points were compared to the starting point activity. Physical stability results are represented in tables 3-11.

TABLE 3
Stability of enzyme compositions comprising the sterol esterase from
Melanocarpus albomyces, 0.04% (w/w) 1,2-benzisothiazolin-3-one as
preservative, sodium citrate as pH buffer, and the
polyhydroxy compound in the
first column. The plus sign marks stable
clear liquid compositions fulfilling
commercial liquid product quality criteria,
whereas the minus sign marks unstable
compositions that developed turbidity and/or
precipitation during storage at 37° C.

| | | after 4 | after 24 |
|---|---|---|---|
| Stabilizer component | pH | weeks | weeks |
| None | 4.92 | − | − |
| None | 5.95 | − | − |
| 15% (w/w) propylene glycol | 5.03 | − | − |
| 30% (w/w) propylene glycol | 4.39 | − | − |
| 30% (w/w) propylene glycol | 5.16 | − | − |
| 40% (w/w) propylene glycol | 5.32 | − | − |
| 15% (w/w) propylene glycol and | 5.02 | − | − |
| 15% (w/w) sorbitol | | | |
| 15% (w/w) propylene glycol and | 5.01 | + | − |
| 35% (w/w) sorbitol | | | |
| 15% (w/w) sorbitol | 4.89 | − | − |
| 30% (w/w) sorbitol | 4.85 | + | − |
| 40% (w/w) sorbitol | 4.79 | + | + |
| 50% (w/w) sorbitol | 4.81 | + | + |
| 15% (w/w) glycerol | 5.05 | − | − |
| 40% (w/w) glycerol | 5.00 | + | + |
| 20% (w/w) maltitol | 5.01 | + | − |
| 40% (w/w) maltitol | 5.04 | + | + |
| 20% (w/w) sorbitol and 20% (w/w) maltitol | 5.00 | + | + |
| 20% (w/w) sorbitol and 20% (w/w) glycerol | 4.90 | + | + |
| 25% (w/w) sorbitol and 25% (w/w) glycerol | 4.95 | + | + |
| 20% (w/w) glycerol and 6% (w/w) maltitol | 5.10 | + | − |

According to the data of table 3, compositions comprising the sterol esterase from Melanocarpus albomyces without stabilizer or with propylene glycol as polyhydroxy compound at citrate buffered acidic pH are not physically stable. The data also reveal that 15% sorbitol concentration is not high enough to prevent physical instability in the presence of 15% propylene glycol. However 35% sorbitol in the presence of 15% propylene glycol is already enough to maintain physically stable liquid for 4 weeks. A composition with 30% sorbitol is maintaining physically stable liquid for at least 4 weeks. Compositions with 40-50% sorbitol or glycerol or mixtures thereof (20%+20% or 25%+25%) are long-term stable. According to 4 weeks data maltitol is an equally good stabilizer as sorbitol and glycerol. Maltitol also functions in mixtures with sorbitol or glycerol at high concentrations.

TABLE 4
Stability of enzyme compositions comprising
the sterol esterase from
Chaetomium thermophilum, 0.35% (w/w) sodium benzoate as
preservative, sodium citrate as pH buffer, and the
polyhydroxy compound in the first column.
The plus sign marks stable clear liquid compositions,
whereas the minus sign
marks unstable compositions that developed turbidity and/or
precipitation during storage at 37° C.

| | | after 4 | after 24 |
|---|---|---|---|
| Stabilizer component | pH | weeks | weeks |
| 40% (w/w) propylene glycol | 5.25 | − | − |
| 10% (w/w) mannitol | 4.81 | − | − |
| 50% (w/w) sorbitol | 4.78 | + | + |
| 25% (w/w) sorbitol and | 4.65 | + | + |
| 6% (w/w) mannitol | | | |
| 25% (w/w) glycerol and | 4.77 | + | + |
| 6% (w/w) mannitol | | | |
| 20% (w/w) sorbitol and | 4.80 | + | + |
| 6% (w/w) mannitol | | | |

According to the data of table 4, composition comprising the sterol esterase from Chaetomium thermophilum with 40% propylene glycol at citrate buffered acidic pH are not physically stable. Physically stable compositions have been achieved using similar high concentrations of sorbitol or mixtures of mannitol with sorbitol or glycerol like used in physically stable compositions of the sterol esterase from Melanocarpus albomyces in table 3. This also reveals that mannitol can be an equally good stabilizer as sorbitol, glycerol and maltitol, but due to solubility limitations, mannitol functions in mixtures with other polyhydroxy compounds to maintain good physical stability.

TABLE 5
Stability of enzyme compositions comprising the enzyme with the
synthetic sequence of SEQ ID NO: 3,
0.35% (w/w) sodium benzoate as preservative,
sodium citrate as pH buffer, and the polyhydroxy compound
in the first column. The plus sign marks stable clear
liquid compositions,whereas the minus sign marks
unstable compositions that developed
turbidity and/or precipitation during storage at 37° C.

| | | after 4 | after 24 |
|---|---|---|---|
| Stabilizer component | pH | weeks | weeks |
| 40% (w/w) propylene glycol | 5.42 | − | − |
| 10% (w/w) mannitol | 4.92 | − | − |
| 50% (w/w) sorbitol | 4.89 | + | + |
| 25% (w/w) sorbitol and | 5.11 | + | + |
| 2% (w/w) glycerol | | | |

The results shown in table 5 reveal comparable physical stability of compositions comprising the enzyme with the synthetic sequence of SEQ ID NO: 3 as the other two sterol esterases in the same compositions as presented in the tables 3 and 4, respectively.

TABLE 6
Stability of enzyme compositions comprising the sterol esterase
from Melanocarpus albomyces, 0.35% (w/w) sodium
benzoate as preservative, sodium citrate as pH buffer,
and the polyhydroxy
compound in the first column. The plus sign marks stable
clear liquid compositions, whereas the minus sign marks
unstable compositions that developed turbidity and/or
precipitation during storage at 37° C.

| | | after 4 | after 24 |
|---|---|---|---|
| Stabilizer component | pH | weeks | weeks |
| 6% (w/w) mannitol | 4.82 | − | − |
| 50% (w/w) sorbitol | 4.78 | + | + |
| 50% (w/w) sorbitol | 5.02 | + | + |
| 25% (w/w) glycerol and | 4.93 | + | + |
| 6% (w/w) mannitol | | | |
| 25% (w/w) sorbitol and | 4.82 | + | + |
| 6% (w/w) mannitol | | | |
| 20% (w/w) sorbitol and | 4.78 | + | + |
| 4% (w/w) mannitol | | | |

Physical stability data shown in the tables 3 and 6 reveal analog trends in the presence of both studied preservatives, sodium benzoate and 1,2-benzisothiazol in-3-one

TABLE 7
Stability of enzyme compositions comprising the sterol esterase from
Melanocarpus albomyces, 0.35% (w/w) sodium
benzoate as preservative, sodium
acetate as pH buffer, and the polyhydroxy compound in t
he first column. The plus sign marks stable
clear liquid compositions, whereas the minus sign marks
unstable compositions that developed turbidity and/or
precipitation during storage at 37° C.

| | | after 4 | after 24 |
|---|---|---|---|
| Stabilizer component | pH | weeks | weeks |
| 20% (w/w) propylene glycol | 4.93 | − | − |
| 40% (w/w) propylene glycol | 4.69 | − | − |
| 40% (w/w) propylene glycol | 5.22 | − | − |
| 20% (w/w) sorbitol | 4.70 | + | − |
| 40% (w/w) sorbitol | 4.70 | + | + |
| 20% (w/w) sorbitol and | 4.77 | + | + |
| 20% (w/w) glycerol | | | |

TABLE 8
Stability of enzyme compositions comprising the sterol esterase from
Melanocarpus albomyces, 0.04% (w/w) 1,2-benzisothiazolin-3-one as
preservative, sodium acetate as pH buffer, and the polyhydroxy
compound in the first column. The plus sign marks stable clear liquid
compositions, whereas the minus sign marks unstable compositions
that developed turbidity and/or
precipitation during storage at 37° C.

| | | after 4 | after 24 |
|---|---|---|---|
| Stabilizer component | pH | weeks | weeks |
| 20% (w/w) propylene glycol | 4.90 | − | − |
| 40% (w/w) propylene glycol | 4.41 | − | − |
| 40% (w/w) propylene glycol | 5.18 | − | − |
| 20% (w/w) sorbitol and | 4.68 | + | − |
| 4% (w/w) mannitol | | | |
| 20% (w/w) sorbitol and | 4.69 | + | − |
| 4% (w/w) maltitol | | | |
| 20% (w/w) sorbitol and | 4.69 | + | − |
| 4% (w/w) xylitol | | | |
| 40% (w/w) sorbitol | 3.93 | + | + |
| 40% (w/w) sorbitol | 4.67 | + | + |
| 50% (w/w) sorbitol | 3.93 | + | + |

Physical stability data shown in the tables 3, 6, 7 and 8 reveal analog trends in the presence of both studied buffers, sodium acetate and sodium citrate. Also xylitol, mannitol and maltitol are equivalent when used in a mixture with sorbitol.

TABLE 9
Stability of enzyme compositions comprising the sterol esterase from
Melanocarpus albomyces, 0.04% (w/w) 1,2-benzisothiazolin-3-one as
preservative, no pH buffer, and the polyhydroxy
compound in the first column. The plus sign marks stable
clear liquid compositions, whereas the minus sign marks
unstable compositions that developed turbidity and/or
precipitation during storage at 37° C.

| | | after 4 | after 24 |
|---|---|---|---|
| Stabilizer component | pH | weeks | weeks |
| none | 7.50 | − | − |
| 15% (w/w) propylene glycol | 7.46 | − | − |
| 15% (w/w) propylene glycol | 8.10 | − | − |
| 20% (w/w) propylene glycol | 5.15 | − | − |
| 30% (w/w) propylene glycol | 7.61 | − | − |
| 30% (w/w) propylene glycol | 8.06 | − | − |
| 50% (w/w) sorbitol | 5.00 | + | + |
| 20% (w/w) sorbitol and 4% (w/w) mannitol | 7.55 | + | − |
| 20% (w/w) sorbitol and 4% (w/w) maltitol | 7.65 | + | + |
| 20% (w/w) sorbitol and 4% (w/w) xylitol | 7.69 | + | + |
| 25% (w/w) sorbitol and 25% (w/w) glycerol | 5.01 | + | + |
| 20% (w/w) glycerol and 4% (w/w) mannitol | 7.82 | + | + |
| 20% (w/w) glycerol and 4% (w/w) maltitol | 7.68 | + | + |
| 20% (w/w) glycerol and 4% (w/w) xylitol | 7.86 | + | + |

Physical stability data shown in table 9 reveal trends presented in the previous tables also in the case of non-buffered sterol esterase compositions at pH close to 5 but also at higher pH range 7.5-8.1.

TABLE 10
Stability of enzyme compositions comprising the sterol esterase from
Melanocarpus albomyces, 0.04% (w/w) 1,2-benzisothiazolin-3-one as
preservative, sodium potassium phosphate as pH buffer,
and the polyhydroxy compound in the first column.
The plus sign marks stable clear liquid compositions,
whereas the minus sign marks unstable compositions that developed
turbidity and/or precipitation during storage at 37° C.

| | | after 4 | after 24 |
|---|---|---|---|
| Stabilizer component | pH | weeks | weeks |
| None | 7.58 | − | − |
| 15% (w/w) propylene glycol | 7.75 | − | − |
| 30% (w/w) propylene glycol | 7.90 | − | − |
| 15% (w/w) propylene glycol and | 7.63 | − | − |
| 15% (w/w) sorbitol | | | |
| 25% (w/w) sorbitol and 25% (w/w) glycerol | 7.39 | + | + |

TABLE 11
Stability of enzyme compositions comprising the sterol esterase from
Chaetomium thermophilum, 0.35% (w/w) sodium benzoate as
preservative, sodium potassium phosphate as pH buffer, and the
polyhydroxy compound in the first column. The plus sign marks
stable clear liquid compositions, whereas the
minus sign marks unstable compositions that developed turbidity
and/or precipitation during storage at 37° C.

| | | after 4 | after 24 |
|---|---|---|---|
| Stabilizer component | pH | weeks | weeks |
| 30% (w/w) propylene glycol | 8.11 | − | − |
| 10% (w/w) mannitol | 7.62 | − | − |
| 25% (w/w) sorbitol and 25% (w/w) glycerol | 7.48 | + | + |

Physical stability data shown in the tables 10 and 11 reveal trends presented in the previous tables in sodium potassium phosphate buffered compositions at pH 7.4-8.1.

### Example 3—Experimental Evaluation of Increasing pH on Stability of Enzyme Compositions

In all experiments enzyme materials used for preparing test compositions were concentrates of clarified fermentation broths, containing preservative to prevent microbial growth, from several different fermentations of the same three enzyme proteins as in the previous experiments in example 2. In the test compositions enzyme liquids were not standardized to certain activity level but only stabilized using different stabilizing conditions shown in the following tables. All test compositions were stored for 4 weeks in climate chamber at 20° C. Otherwise storage stability study was done in the same manner as described in the example 2. Physical stability results are represented in table 12.

TABLE 12
Stability of compositions comprising the indicated enzyme and the experimental
conditions presented in the table. Unstable compositions developed turbidity
and/or precipitation immediately at room temperature and/or
during storage at 20° C. The plus
sign marks stable clear liquid compositions, whereas the minus sign
marks unstable compositions.

| | Stabilizing conditions of enzyme | | immediate | |
|---|---|---|---|---|
| Enzyme * | compositions not standardized by activity | pH | observations | after 4weeks |
| MA | 0.04% (w/w) 1,2-benzisothiazolin-3-one | 5.56 | − | − |
| MA | 0.04% (w/w) 1,2-benzisothiazolin-3-one | 7.48 | + | − |
| MA | 30 % (w/w) propylene glycol, | 5.91 | − | − |
| | 0.04% (w/w) 1,2-benzisothiazolin-3-one | | | |
| MA | 30 % (w/w) propylene glycol, | 7.50 | − | − |
| | 0.04% (w/w) 1,2-benzisothiazolin-3-one | | | |
| MA | 0.35 % (w/w) sodium benzoate | 5.98 | − | − |
| MA | 0.35 % (w/w) sodium benzoate | 7.60 | − | − |
| MA | 30 % (w/w) propylene glycol, | 6.22 | − | − |
| | 0.35 % (w/w) sodium benzoate | | | |
| MA | 30 % (w/w) propylene glycol, | 7.60 | − | − |
| | 0.35 % (w/w) sodium benzoate | | | |
| CT | 0.35 % (w/w) sodium benzoate | 5.92 | − | − |
| CT | 0.35 % (w/w) sodium benzoate | 7.47 | − | − |
| CT | 30 % (w/w) propylene glycol, | 6.22 | − | − |
| | 0.35 % (w/w) sodium benzoate | | | |
| CT | 30 % (w/w) propylene glycol, | 7.44 | − | − |
| | 0.35 % (w/w) sodium benzoate | | | |
| SEQ3 | 0.35% (w/w) sodium benzoate | 5.80 | − | − |
| SEQ3 | 0.35% (w/w) sodium benzoate | 7.49 | − | − |
| SEQ3 | 30% (w/w) propylene glycol, | 6.10 | − | − |
| | 0.35% (w/w) sodium benzoate | | | |
| SEQ3 | 30% (w/w) propylene glycol, | 7.61 | − | − |
| | 0.35% (w/w) sodium benzoate | | | |

* MA for Melanocarpus albomyces sterol esterase, CT for Chaetomium thermophilum sterol esterase, and SEQ3 for the enzyme with the synthetic sequence of SEQ ID NO: 3

As the examples in Table 12 reveal, increasing pH was not sufficient for physical stability. In one experiment with the composition comprising the sterol esterase from Melanocarpus albomyces and 1,2-benzisothiazolin-3-one as preservative appearance of the liquid was improved by adjusting pH from 5.6 to 7.5 but the composition is clear only at the time of preparing and is not physically stable. Also high pH like 7.5 has overall stability decreasing effect as seen in the following table 13 showing activity drop of standardized enzyme compositions at pH 5 and 7.5.

### Example 4—Experimental Evaluation of Liquid Enzyme Composition Stabilities Measured as Remaining Activity

Materials and methods were as described in example 1 and 2.

TABLE 13
Stability of compositions comprising enzymes, polyhydroxy compounds,
antimicrobial preservatives and pH buffers as indicated in the table. Stability was
evaluated in this example as remaining enzyme activity after storage. Enzyme activities
were measured as described in example 1 and normalized to the respective enzyme
activity in the beginning. Thus, enzyme activities start at 100%, and then during storage
at 37° C. decrease depending on the enzyme compositions, which comprise a sterol
esterase at standardized activity level range from 1200 to 1900 SEU/g, as antimicrobial
preservative 0.35% (w/w) sodium benzoate or 0.04% (w/w) 1,2-benzisothiazolin-3-one,
which were abbreviated in the table as benzoate and isothiazolinone, respectively, and
different polyhydroxy compounds as indicated in the table.

| Composition | | | | | Enzyme activity | | |
|---|---|---|---|---|---|---|---|
| | Stabilizer | | | | | after 4 | after 8 |
| Enzyme* | component | Preservative | Buffer | pH | at start | weeks | weeks |
| MA | 30 % propylene | isothiazolinone | no buffer | 7.61 | 100% | 33% | 17% |
| | glycol | | | | | | |
| MA | None | isothiazolinone | no buffer | 7.50 | 100% | 10% | 4% |
| MA | None | isothiazolinone | citrate | 4.92 | 100% | 28% | 17% |
| MA | 50% sorbitol | benzoate | citrate | 5.02 | 100% | 63% | 52% |
| SEQ3 | 40% propylene | benzoate | citrate | 5.42 | 100% | 3% | 0% |
| | glycol | | | | | | |
| SEQ3 | 50% sorbitol | benzoate | citrate | 4.89 | 100% | 89% | 80% |
| SEQ3 | 25% sorbitol and | benzoate | citrate | 5.11 | 100% | 96% | 54% |
| | 25% glycerol | | | | | | |
| CT | 30% propylene | benzoate | phosphate | 8.11 | 100% | 0% | 0% |
| | glycol | | | | | | |
| CT | 40% propylene | benzoate | citrate | 5.25 | 100% | 1% | 0% |
| | glycol | | | | | | |
| CT | 25% sorbitol and | benzoate | citrate | 4.65 | 100% | 80% | 74% |
| | 6% mannitol | | | | | | |
| CT | 25% sorbitol and | benzoate | phosphate | 7.48 | 100 % | 51% | 43% |
| | 25% glycerol | | | | | | |

*MA for Melanocarpus albomyces sterol esterase, CT for Chaetomium thermophilum sterol esterase, and SEQ3 for the enzyme with the synthetic sequence of SEQ ID NO: 3.

A remaining enzyme activity of more than 50% after 4 weeks storage at 37° C. demonstrates a stable enzyme composition. Also more than 40% remaining enzyme activity after 8 weeks storage at 37° C. demonstrate a stable enzyme composition.

The disclosure above has provided by way of non-limiting examples of particular implementations and embodiments of the invention a full and informative description of the best mode presently contemplated by the inventors for carrying out the invention. It is however clear to a person skilled in the art that the invention is not restricted to details of the embodiments presented above, but that it can be implemented in other embodiments using equivalent means without deviating from the characteristics of the invention.

Furthermore, some of the features of the above-disclosed embodiments of this invention may be used to advantage without the corresponding use of other features. As such, the foregoing description should be considered as merely illustrative of the principles of the present invention, and not in limitation thereof. Hence, the scope of the invention is only restricted by the appended patent claims.

1. A solution stable enzyme composition comprising:
(a) an enzyme component comprising an enzyme characterized by having the amino acids sequence WGESAG and a catalytic triad composed of S, H and E/D;
(b) a stabilizer component comprising an organic sugar alcohol that has three or more hydroxyl groups per molecule, and wherein the stabilizer component comprises at least one fifth of the total weight of the enzyme composition;
(c) an optional antimicrobial preservative component preventing growth of a microorganism; and
(d) water having dissolved therein said components (a), (b) and (c);
wherein the enzyme component of the solution stable enzyme composition remains in solution for at least 4 weeks upon storage at 37° C.

2. The solution stable enzyme composition according to claim 1, wherein said enzyme is a hydrolase characterized by having enzyme activity on a sterol ester, preferably an ester of sterol with a fatty acid, more preferably an ester of cholesterol with linoleic acid.

3. The solution stable enzyme composition according to any of claims 1-2, wherein said enzyme is a thermostable enzyme that retains at least 50% enzyme activity after incubation in an aqueous composition or aqueous environment or aqueous solution at 50° C., preferably 60° C., more preferably 70° C., most preferably 75° C., for at least 5 minutes.

4. The solution stable enzyme composition according to any of claims 1-3, wherein said enzyme is a fungal sterol esterase, preferably a thermophilically fungal sterol esterase.

5. The solution stable enzyme composition according to any of claims 1-4 wherein the enzyme is a sterol esterase and the catalytic triad is composed of S, H and E.

6. The solution stable enzyme composition according to any of claims 1-5, wherein said enzyme is a sterol esterase encoded by a gene from the genome of a thermophilic fungus selected from the group consisting of Scytalidium thermophilum, Myceliophthora thermophila, Thielavia terestris, Corynascus thermophilus, Myriococcum thermophilum, Thermomyces stellatus, Thielavia australiensis, Malbranchea cinnamomea, Melanocarpus albomyces and Chaetomium thermophilum and fragments thereof and conservative alterations thereof.

7. The solution stable enzyme composition according to any of claims 1-6, wherein said enzyme has at least 60%, preferably at least 70%, more preferably at least 75%, most preferably at least 80% sequence identity with a sterol esterase from SEQ ID NO: 1 or SEQ ID NO: 2.

8. The solution stable enzyme composition according to any of claims 1-7, wherein said sugar alcohol has four or more, preferably five or more, more preferably six or more hydroxyl groups per molecule.

9. The solution stable enzyme composition according to any of claims 1-8, wherein said sugar alcohol is selected from the group consisting of sorbitol, mannitol, maltitol, xylitol and glycerol.

10. The solution stable enzyme composition according to any of claims 1-9 comprising antimicrobial preservative selected from isothiazolinones, preferably selected from the group consisting of 1,2-benzisothiazolin-3-one, 2-methyl-4-isothiazolin-3-one, 5-chloro-2-methyl-4-isothiazolin-3-one, 2-octylisothiazolin-3-one and 4,5-dichlor-2-octylisothiazolin-3-one.

11. The solution stable enzyme composition according to any of claims 1-10, wherein said stabilizer component comprises at least one fourth, preferably at least one third, of the total weight of the enzyme composition

12. The solution stable enzyme composition according to any of claims 1-11 comprising antimicrobial preservative selected from the group consisting of benzoic acid, 4-hydroxy-benzoic acid, 2-hydroxy-benzoic acid, and salts thereof and esters thereof.

13. The solution stable enzyme composition according to any of claims 1-12, wherein the preservative is active against at least one microorganism selected from the group consisting of acid-tolerant bacteria, molds and yeasts.

14. The solution stable enzyme composition according to any of claims 1-13, wherein the composition is essentially free from polyethylene glycol p-alkyl-phenyl ethers, preferably free from all nonionic surfactants, more preferably free from all surfactants.

15. The solution stable enzyme composition according to any of claims 1-14, wherein the composition is essentially free from serine protease inhibitors, preferably free from all protease inhibitors.

16. A use of the solution stable enzyme composition according to any of claims 1-15 in the manufacture of pulp.

17. A use of the solution stable enzyme composition according to any of claims 1-15 in the manufacture of paper.
